(19) United States
(12) Patent Application Publication
(10) Pub. No.: US 2005/0249724 A1
(43) Pub. Date: Nov. 10, 2005

(54) Extracorporeal stablised expanded bed adsorption method for the treatment of sepsis

(75) Inventors: Allan Otto Fog Lihme, Birkerod (DK); Peter M.H. Heegaard, Copenhagen (DK)

Correspondence Address: FOLEY AND LARDNER;SUITE 500, 3000 K STREET NW, WASHINGTON, DC, 20007, US

(73) Assignee: Upfront Chromatography A/S

(21) Appl. No.: 10/520,685

(22) PCT Filed: Jul. 9, 2003
(86) PCT No.: PCT/DK03/00483

§ 371 Date: May 27, 2005

(30) Foreign Application Priority Data
Jul. 11, 2002 (DK) PA 2002 01091

(51) Int. Cl. A61K 39/395

(52) U.S. Cl. 424/140.1

(57) ABSTRACT

The present invention provides an extracorporeal adsorption method for removing harmful substances from blood in a way that is practicable in everyday clinical practice and applicable for the timely intervention to present the development of sepsis. Said extracorporeal adsorption method being effected by an adsorption column assembly where the adsorption column assembly comprising a column and an adsorption medium in the form of particles. The sedimented volume of said particles being at the most 80% of the volume of the column.

### FIELD OF THE INVENTION

The present invention concerns a method for the treatment of sepsis by specific depletion of harmful substances from the circulating blood of a patient by means of subjecting the patient's blood to extracorporeal adsorption through a stabilised fluidised bed of an adsorption medium characterised by having specific affinity towards harmful substances promoting sepsis, such as those related to Gram-negative and Gram-positive bacteria. In one particular embodiment the method is applied to the treatment of Gram-negative sepsis by employing a stabilised fluidised bed of an adsorption medium having specific affinity against the endotoxin (lipopolysaccharlde (LPS) portion) of Gram-negative bacteria.

### BACKGROUND OF THE INVENTION

Sepsis (bacteremia, septicemia, septic syndrome) is defined herein as the clinical consequence of a bacterial infection in which bacteria are found in the bloodstream (Gale Encyclopedia of Medicine, Gale Research 1999). The multiple symptoms of sepsis can be ascribed to imbalanced (exaggerated) immune and inflammatory host reactions on a systemic scale towards highly inflammatory bacterial cell wall components. The end result is tissue damage and, ultimately multiple organ dysfunction with a high degree of morbidity and mortality. Contributing to the development of this syndrome is the concomitant over-activation of the coagulation system and the suppression of fibrinolysis. One common advanced clinical situation in sepsis is septic shock in which severe hypotension is seen.

Sepsis and septic shock are life-threatening complications and are promoted by a high load of infectious pathogen, inability to cope with the infection by the immune system and inadequate or delayed treatment with antibiotics. Patient groups with compromised or diminished immune competence (premature neonates, the elderly, patients undergoing immuno-suppressive therapy, the critically III, etc.) have high mortality (up to 60%) while for otherwise healthy individuals the mortality is around 5% for uncomplicated sepsis and 40% for more advanced sepsis (septic shock). Overall, sepsis mortalities lie in the range of 30-50%. The condition remains a problem, and even an increasing problem among hospitalised patients, especially in critical care units and in US sepsis has increased by an average of 8.7% each year over the past 22 years Current estimates of the incidence of severe sepsis is 700.000 cases pr. year (sepsis associated with acute organ dysfunction) in the US alone (www.sepsis.com/epidemiology.jsp). This increase in the incidence of sepsis is probably due to the rise in spread of antibiotic resistance, rendering preventive antibiotic treatment inefficient and is also an effect of improvements in survival rates of patients predisposed to sepsis and a result of the general “ageing” of populations in the western part of the world. In-hospital deaths due to sepsis reached 120,491 in 2000; in 1979 the number was 43,579 (US, Martin et al., 2003, New England Journal of Medicine 348, 1546-1554).

It has been estimated that 70% of infections leading to septic shock in human patients are caused by Gram-negative bacteria (acting through endotoxin (lipopolysaccharide, LPS)) and 30% by Gram-positive bacteria (acting through cell wall components (peptidoglycans, lipoteichoic acid) and exotoxins) (Gutierrez-Ramos & Bluethmann, 1997, Molecules and mechanisms operating in septic shock: lessons from knock-out mice, Immunology Today 18, 329-334). Purified LPS can by itself create most of the sepsis syndrome.

The effects of these toxins are mediated primarily by tumor necrosis factor alpha (TNFα) and other cytokines, including interleukin 1 (IL-1), IL-6 and IL-8, being massively released by monocytes, macrophages and other leukocytes (most effects are mediated by macrophages), upon exposure to such toxins. These cytokines in turn have profound effects on other cells of the immune system as well as on other types of cells (Gutierrez-Ramos & Bluethmann, 1997, Molecules and mechanisms operating in septic shock: lessons from knock-out mice, Immunology Today 18, 329-334)

“Endogenous” endotoxin may occur in cases of compromised mucosal barriers and may lead to sepsis-like states in such patients.

While the triggering components of Gram-positive bacteria have not been precisely defined and may include peptidoglycans, lipoteichoic acid and certain proteinaceous exotoxins, it is quite clear that endotoxin is responsible for the initial triggering of Gram-negative sepsis. Gram-negative endotoxin activity resides in lipopolysaccharides (LPS) (Rietschel and Brade, Bacterial Endotoxins, Sci. American, August 1992, 26-33) which are the main component of the outer membrane of Gram-negative bacteria. As is well-known to anyone skilled in the field, LPS has very dramatic biological effects owing to the potent inflammatory and immunostimulating properties of the lipid A-part of the LPS-molecule and LPS is generally believed to contribute profoundly to the pathogenesis of Gram-negative bacterial infections and to other diseases. The actions of LPS in biological systems are very complex. First of all, LPS really does not exist as a free molecule in solution in e.g. the blood stream but instead is organised as micelles held together by the highly hydrophobic lipid A part or is bound to cell membranes (the Gram-negative bacterial cell membrane or a host cell membrane) or host cell membrane receptors through the lipid A part. Host cell membrane receptors are typically proteins and also a number of soluble, LPS-binding proteins have been described. Examples of such mammalian proteins include LPS-binding protein (LBP), the monocyte/macrophage marker protein CD14, BPI (bacterial permeability increasing protein), and other endotoxin-binding proteins (for example NEP and CAP-18). In mammals, a very important group of LPS-receptors are the Toll-like receptors, of which TLR4 is regarded as the main LPS receptor and TLR2 as a minor receptor also having other microbial products as ligands, notably lipoteichoic acid and peptidoglycan from Gram-positive bacteria. TLR4 is considered to be indispensable for the activation of cells by LPS. Some of the receptor proteins may also exist as soluble entitles. Such soluble receptors are believed to act as transport molecules transporting LPS from bacteria to host cells. Blocking CD14 or LPS-binding protein (LBP) results in protection against LPS-toxicity (Gutierrez-Ramos & Bluethmann, 1997, Molecules and mechanisms operating in septic shock: lessons from knock-out mice, Immunology Today 18, 329-334). The host cells interacting with LPS include monocytes, macrophages and granulocytes and they are normally very efficient in removing LPS from the blood stream, the problem being, however, their exaggerated activation by LPS. It is currently believed that LPS is bound as a LPS-LBP complex by CD14 and TLR4 on the surface of macrophages, resulting in massive actvation of these cells. A small protein called MD-2 is also believed to be involved in the actual signal transduction; while direct binding between LPS and MD2 has been demonstrated by Mancek, M. et al. to provide direct binding between LPS and TLR-4 by itself has yet to be demonstrated, although TLR4 is binding to the CD14/LPS complex. The clinical outcome of LPS challenge in a patient is the result of the reactions and mediators produced by a whole range of different cell types reacting to LPS as well as depending very much on the timing of these individual cell responses. One characteristic feature of sepsis is the rapidity of these reactions—the first clinical effects to venous administration of LPS occur within minutes. Another consistent finding after experimental challenge with LPS is the induction of tolerance to LPS which is probably mediated by carbohydrate-specific antibodies (late tolerance) but also comprises a non-characterised component (early tolerance). One of the typical, early host responses to LPS is the acute phase protein response in which certain serum proteins react quickly and quite dramatically by considerable increases in their serum concentrations; the fastest acute-phase proteins will be on the rise a few hours after administration of LPS and will reach serum concentrations 100 times their normal concentrations during this response; LPS is one of the most potent acute phase response inducers. In man, these early and heavily induced acute phase proteins include C-reactive protein and serum amyloid A.

In addition to removal by macrophages, LPS is also removed by binding to high-density lipoprotein particles followed by transport to and breakdown by the liver.

Three points of intervention have traditionally been considered, namely
removal of the infection by appropriate antimicrobial therapy or—in the case of a localised infection—surgical drainage
treatment of resulting cardiovascular and multiorgan disturbances
inhibition of toxic mediators

Current principles for the treatment of sepsis are based on identification of the causative organism(s) and administering the corresponding appropriate antibiotics. A significant drawback to this approach is the time needed before the basis for a decision has been established by identifying the causative organism(s), and thus broad-spectrum antibiotic treatment will often have to be initiated first to cope with the speed of the clinical development of the syndrome. Such antibiotic treatments are normally intravenous and demand hospitalisation and are often inadequate.

Early antimicrobial therapy, and detailed monitoring of organ functions in intensive care units and corresponding treatment centred on counteracting hypotension (hemodynamic support) are pivotal. Toxic mediator inhibition, e.g. by administration of anti-endotoxin antibodies has generally failed in the clinical setting, where early initiation of this kind of treatment is all-important. The use of inhibitors against host inflammatory cytokines (especially tumor necrosis factor alpha (TNFα) and interleukin 1) have also been investigated as they have the benefit of not being confined to either gram negative or gram positive sepsis. However, the potential adverse side effects arising from the use of inhibitors against such cytokines are potentially serious as these cytokines also participate in a multitude of beneficial, inflammatory and immunological defence reactions of the host. Also, these kinds of inhibitors have generally failed to reduce mortality in large clinical studies of sepsis. This is probably due to the fact that removal of the products of LPS-activation does not remove the substance causing the problem.

Three possible therapeutic targets in the circulation of the blood for intervention against sepsis (sepsis treatment points) present themselves:
1. LPS and cells carrying LPS, including host monocytes and bacteria
2. Responding host cells (macrophages)
3. Factors of the host response and the cells responding to these factors, e.g. TNF and TNF-receptor-bearing cells. IL-1 also plays a major role.

For treatment of sepsis, methods directed towards removal of unwanted components from patients' blood are thus very relevant. Such procedures include plasma exchange therapy in which the patient's plasma is replaced partially with a plasma substitute free of the harmful component(s). In such a procedure there is a need for expensive, fully certified plasma or plasma fractions; also, potentially beneficial components are removed from the patient and there are all the dangers associated with blood and blood product transfusion, e.g. of transferring infections (especially virus, prions etc.). Other procedures comprise the use of membranes to filtrate the blood but they lack selectivity and concurrently remove proteins that need to be replaced.

The possibility of treating sepsis by extracorporeal adsorption methods has been reported previously (see e.g. Jaber & Pereira, 1997, Extracorporeal Adsorbent-based strategies in sepsis, Am. J. Kidney Diseases 30, S44-556). Such methods comprise non-specific adsorption methods (including for example ion exchange resins, activated charcoal, immobilised cholestyramine) and specific adsorbents (for example Polymyxin B-Sepharose). Soft gels with specific affinity ligands give good selectivity but lead to difficulties with clogging and poor flow rates when used to handle viscous, particulate suspensions like blood. On the other hand, harder materials like polystyrene-derivatised fibers offers good mechanical stabilities but have low capacities

Currently known extracorporeal methods show moderate efficiency for removal of small, water-soluble substances (Bellomo et al., 2001, Blood purification in intensive care, Contrib. Nephrol. 132, 367-374), but larger molecules are only removed to a limited extent. There is a need for improved technology in combination with the best targets.

As LPS constitutes a central disease-mediator in Gram-negative sepsis, specific affinity ligands with specificity for LPS have naturally been attractive for use as LPS-depleting or inhibiting substances with a therapeutic potential and such ligands are also most useful for incorporation into adsorption media used for extracorporeal adsorption.

One such class of affinity specific molecules are antibodies; this was further underlined by the finding that high levels of naturally occurring antibodies against core-saccharide structures of LPS (see FIG. 1) were associated with a better prognosis of sepsis than low levels and could be used to define which patients could benefit from therapy by passive administration of such antibodies (Strutz, et al., 1999, Relationship of antibodies to endotoxin core to mortality in medical patients with sepsis syndrome, Int. Care Med. 25, 435-444). Such antibodies have been purified from donor blood and were shown to protect animals from E. coli sepsis and were proposed to be of use in passive therapy of sepsis patients with Gram-negative bacteraemia (Barclay, G. R., 1999, Endotoxin-core antibodies: time for a reappraisal?, Int. Care Med. 25, 27-29). These same authors were however led to conclude that “there are currently no intervention treatments indicated and available for general use other than the standard treatments”, including the realisation that none of the anti-LPS antibodies tested so-far have stood up to clinical testing, examples including HA-1A (humanised monoclonal antibody) and E-5 (murine monoclonal). These authors, however, share the view with others (Cross et al., 1999, Immunotherapy of sepsis: flawed concept or faulty implementation”) that the utility of antibodies for treatment of sepsis has not been conclusively disproved but just awaits its right mode of employment.

No monodonal antibodies have been of use, maybe because the exact spatial structure of common LPS core structures are highly dependent on the oligosaccharide units, their glycosidic bonds and positions and number of substituent (phosphate, sulfate etc.). Thus supposedly cross-reacting epitopes in reality may not cross-react and there has apparently also been a lack of focus on the functional affinities of such antibodies. Also the pharmacokinetics of injected antibodies is an important point to consider when attempting treatment by administering therapeutic antibodies directed against LPS directly by injection into patients.

Antibodies with proven cross-reactivity with a number of important enterobacterial LPS-types and directed against not lipid A but against conserved core-saccharide structures, and, most importantly with endotoxin-neutralising and protective activity have become available and were proposed for therapeutic applications against sepsis (Barclay, G. R., 1999, Endotoxin-core antibodies: time for a reappraisal?, Int. Care Med. 25, 27-29). An example of such an antibody is a humanised monoclonal (WN1 222-5) which is currently being tested clinically.

Another interesting compound with specific affinity for LPS is Polymyxin B and it's analogues (e.g. colistin). These substances are well-known and fully characterised amphipathic, cationic cyclic peptide antibiotics of the structure depicted in FIG. 2 (Merck Index, Vol. 13, entry 7656). They have detergent-like properties and have the ability to bind Gram-negative lipopolysaccharides, irrespectively of the bacterial species of origin. They specifically bind to the lipid A part, presumably by a combination of ionic and hydrophobic interactions. The exact binding site in LPS has not been defined but is likely to include the negatively charged phosphate groups in lipid A and/or the acidic KDO-monosaccharides of the inner core of LPS. Studies with free LPS have indicated that Polymyxin B is able to disrupt LPS-LPS-micelles thereby exposing antibody-binding epitopes in LPS (Barclay, http://freespace.virgin.net/r.barclay/endocela.htm).

Although these compounds are highly antibiotic they are also highly nephrotoxic and neurotoxic and thus are rarely used for direct administration to patients. Like other antibiotics they may even promote sepsis by facilitating release of LPS from bacteria upon killing them. Polymyxin B was also recently shown to stimulate peripheral blood mononuclear cells to produce tumor necrosis factor alpha upon incubation in vitro (Jaber et al., 1998, “Polymyxin-B stimulates tumor necrosis factor-alpha production by human peripheral blood mononuclear cells, Int. J. Artificial Organs 21, 269-273). However, such compounds would be very useful as specific affinity ligands in extracorporeal adsorption methods. A big number of other peptidic LPS-binding substances have been described, including naturally occurring cationic antibacterial peptides (Hancock, R. E., 2001, “Cationic peptides: effectors in innate immunity and novel antimicrobials”, Lancet Inf. Dis, 1, 156.164) and other peptides mimicking Polymyxin B (Rustici et al., 1993, “Molecular mapping and detoxification of the lipid A binding site by synthetic peptides”, Science 259, 361-365).

Shoji et al. (Shoji, H., et al., 1998, Therapeutic Apheresis 2, 3-12) specifically teaches the use of Polymyxin B immobilised on polystyrene fibres in a fibre cartridge design and shows the ability of a direct hemoperfusion procedure using this unit to achieve therapeutic improvements in a number of different parts of the clinical picture representing sepsis/septic shock. The device was connected via a blood pump with the femoral vein of the patient. It was found that the capacity of this adsorbent to bind LPS was correlated to the number of free primary amino groups available in the immobilised Polymyxin B-molecules, but generally it was possible to immobilise Polymyxin B through some of it's primary amino groups and still retain LPS-binding capacity. The same approach was taken by Nemoto et al. (Nemoto, H., et al., 2001, Blood Purif. 19, 361-369) who further tested the device on different groups of patients and concluded that treatment was beneficial (improvement of survival rates) when applied at early stages of sepsis but had no effect on severe sepsis. Hemoperfusion was carried out for 2-4 hours at 80-100 ml/min using nafamostat mesilate (30-50 mg/h) or heparin as anticoagulant. Platelet counts were slightly decreased after this treatment due to unspecific adsorption of these cells in the cartridge but this was judged to be a not-so-serious adverse effect.

Continuous therapy is beneficial for blood purification methods as it reduces hemodynamic instability, prevent and treat fluid overload, and offers superior control of uremia. However, continuous methods have until now been hampered by technical difficulties, especially relating to clogging/fouling of the adsorbent devices and also specifically relating to the inferior capacity of such devices, one example being the relatively low capacity of hollow fibre devices as the ones described above. For example bead-shaped adsorbents have much higher surface areas than hollow fiber-based adsorbent materials.

The ideal adsorbent for use in hemoperfusion or plasma perfusion with extracorporeal circulation (plasmapheresis) is sufficiently stable to withstand high flow rates of viscous fluids containing suspensions of cells such as blood. This contrasts somewhat with the need for conventional adsorbents to be hydrophilic and porous in order to accommodate solute molecules and adsorbents and allowing their surface interaction on inside surfaces inside pores of the particles, in order to achieve a satisfactory binding capacity. Another demand for adsorbents useful for treating blood is that the back-pressure observed with high flow rates of blood through the adsorbent is negligible to an extent that prevents shearing of the various fragile cells being suspended in blood.

Examples of extracorporeal methods and adsorbents include specific adsorption of lipoproteins on porous, hard particles (U.S. Pat. No. 4,656,261). This was only shown to work however in a stirred batch experiment. In another example of prior art a method based on plastic (particles, film or hollow fibre) coated with albumin (U.S. Pat. No. 6,090,292) was disclosed. This method takes advantage of the fact that albumin can be used as an ligand for detoxifying blood or plasma with a big number of important bacterial toxins and medication substances. There is no teaching, however on how to construct a perfusable packing from 10-500 micrometer (diameter) particles and instead examples of batch-wise adsorption of whole blood are disclosed, while examples with packed columns comprise plasma pumped at a flow rate of only 2 ml/min and heparinised whole blood perfused at 0.5 m/min only. U.S. Pat. No. 5,041,079 teaches removing agents for treatment of patients harbouring the human immunodeficiency virus and the use of plasma instead of blood is recommended. U.S. Pat. No. 5,258,503 discloses components in an extracorporeal system for removing autoantibodies, said system incorporating filters to separate particulate material from the soluble components of blood and using porous and hard particles as adsorbents. In U.S. Pat. No. 4,865,841 describes removing unwanted antibodies by contacting them with immobilised antigen (silica) in order to allow therapeutic immunotoxins to exert their effect. Removal was affected by passage of plasma (prepared by hollow fibre filtration) which could be pumped at 20-25 ml/min. Other devices for keeping particles into suspension include the Taylor-Couette flow device by Ameer et al. (Biotech. Bioegn. 62, 602-608, 1999) which is however also characterised by a substantial problem with shear which led to a high degree of hemolysis.

Continuous venovenous circuits are operated by appropriate peristaltic pumps and are much preferred to arteriovenous circuits because cannulation of arteries is a difficult and dangerous process. An anticoagulant (heparin, citrate) is normally used to prevent dotting and causes no problem for the patient as long as concentrations are kept low e.g. by observing the activated clotting time of the blood and adjusting the anticoagulant concentration accordingly.

### BRIEF DESCRIPTION OF THE INVENTION

The present invention provides a means for extracorporeal treatment of blood in a way that is practicable in everyday clinical practice and applicable for the timely intervention to prevent the development of sepsis.

Another aspect of the present invention is to provide extracorporeal therapeutic and prophylactic devices based on efficient adsorption of bacterial toxins from blood.

It is characteristic for stabilised fluidised bed adsorption processes that an expansion of the bed occurs upon application of a liquid, typically in an upward flow through the bed. The space between the particles of the adsorption medium, the void volume, is thereby increased allowing large or bulky molecules (e.g. bio-macromolecular entities) contained in the sample to pass through without clogging the column. It has been found that this property makes fluidised bed adsorption particular suitable in connection with separation of specific components from blood which comprise many different components, e.g. blood cells. Furthermore, the space between the particles created by the upward flow allows the passage of cells through the stabilised fluidised bed at a high flow rate without experiencing shear that may damage the cells. Also, in a stabilised fluidised bed the liquid is passed through the column as a plug flow substantially without turbulence and back-mixing.

Optimal performance of the disclosed stabilised fluidised bed capture of bacterial toxins from blood is further ensured by providing a very large surface area of the particles to accomplish an efficient and high capacity adsorption process combined with a large density difference between the density of the blood and the density of the particles to accomplish an acceptable flow rate through the column.

In one aspect, the present invention provides the use of an adsorption column assembly for the preparation of a medical device for the treatment of sepsis caused by Gram-negative bacteria in a mammal by extracorporeal adsorption, said adsorption column assembly comprising a column and an adsorption medium in the form of particles, the sedimented volume of said particles being at the most 80% of the volume of the column, said particles being characterised by carrying an affinity specific molecule with a specific affinity for the LPS portion of said Gram-negative bacteria.

In another aspect, the present invention provides the use of an adsorption column assembly for the preparation of a medical device for the treatment of sepsis caused by Gram-negative or Gram-positive bacteria in a mammal by extracorporeal adsorption, said adsorption column assembly comprising a column and an adsorption medium in the form of particles, the sedimented volume of said particles being at the most 80% of the volume of the column, said particles being characterised by carrying an affinity specific molecule with a specific affinity for
i) the LPS portion of said Gram-negative bacteria, and/or
ii) Gram-positive bacteria or harmful substances derived from said Gram-positive bacteria.

In a further aspect, the present invention provides a method for the treatment of sepsis caused by Gram-negative in a mammal by extracorporeal adsorption, said extracorporeal adsorption being effected by an adsorption column assembly, said adsorption column assembly comprising a column and an adsorption medium in the form of particles, the sedimented volume of said particles being at the most 80% of the volume of the column, said particles being characterised by carrying an affinity specific molecule with a specific affinity for the LPS portion of said Gram-negative bacteria, said method comprising the steps of
a) obtaining blood from said mammal,
b) treating the obtained blood by passing the blood through the adsorption column assembly at such a flow rate that a fluidised bed of the particles is formed, and
c) reinfusing the treated-blood into the same mammal.

In a still further aspect, the present invention provides a method for the treatment of sepsis caused by Gram-negative or Gram-positive bacteria in a mammal by extracorporeal adsorption, said extracorporeal adsorption being effected by an adsorption column assembly, said adsorption column assembly comprising a column and an adsorption medium in the form of particles, the sedimented volume of said particles being at the most 80% of the volume of the column, said particles being characterised by carrying an affinity specific molecule with a specific affinity for
i) the LPS portion of said Gram-negative bacteria, and/or
ii) Gram-positive bacteria or harmful substances derived from said Gram-positive bacteria, said method comprising the steps of
   a) obtaining blood from said mammal,
   b) treating the obtained blood by passing the blood through the adsorption column assembly at such a flow rate that a fluidised bed of the particles is formed, and
   c) reinfusing the treated blood into the same mammal.

### BRIEF DESCRIPTION OF THE DRAWINGS

FIG. 1 illustrates the general structure of gram-negative bacterial lipopolysaccharides. The zigzag horizontal lines in lipid A represent fatty acids, typically C12 to C16, bound as esters or amides to the two glucosaminyl residues. GlcN: glucosamine, GlcNAc: N-acetylglucosamine, Glc: Glucose, Gal: Galactose, Hep: Heptose, KDO: 2-keto-3-deoxyactonic acid, PO₄²⁻: Phosphate (is typically present elsewhere in the core sugars also). After: Rietschel et al., 1993, The chemical structure of bacterial endotoxin in relation to bioactivity, Immunobiology 187, 169-190.

FIG. 2 illustrates the general structure of Polymyxin B (after Merck Index Vol. 13, entry 7656). Dab: diaminobutyric acid, Thr: threonine, Phe: Phenylalanine, Leu: Leucine, R is a fatty acid attached to the α-amino group of the N-terminal Dab. Positive charges carried by free primary amino groups are indicated. Arrows indicate the direction of the decapeptide chain.

FIG. 3 illustrates the principle of continuous extracorporeal adsorption. Shown in the figure is a vessel “a” continuously receiving blood from the patient and connected to a stabilised fluidised bed column (“b”) through a valve which may be closed or open. The blood stream is applied in an upward direction from the bottom of the stabilised fluidised bed and is then led from the top of the column through another valve to vessel “c” which continuously delivers blood back into the patient. The “valves” may be in the form of pump for continuous or intermittent distribution of blood to the column, or a separate pump (not shown) may be utilised

FIG. 4 illustrates the set-up of a stabilised fluidised bed column using commercially available equipment (Upfront Chromatography A/S, 7010-0000—diameter 1.0 cm, height 50 cm). The equipment comprises a vertical glass column held in place by a foot plate also containing tube connector for the inlet fluid. Furthermore the glass column is equipped with an outlet fluid connector at the top of the column. (A) Shows the column disassembled in its transport container. (B) Shows the assembled column.

FIG. 5 illustrates biotin-coupled conglomerate particles with a core of glass particles stained by DAB (+biotin) and nonstained (−biotin). Conglomerate adsorbent particles with cores of glass particles, either underivatised (A) or derivatised with biotin as the ligand (B). Both types of particles were used for stabilised fluidised bed chromatography of EDTA-stabilised human blood spiked with avidin-peroxidase as described in Example 2. After chromatography and wash a sample of each type of adsorbent particles were subjected to DAB-staining to reveal the presence of peroxidase activity on the surface of the particles. Peroxidase activity gives rise to a brown (dark in the black & white figure) coloration of the particles as seen for the biotin-coupled particles but not for the non-derivatised particles.

FIGS. 6a and 6b show conglomerate adsorbent particles (particles of agarose with cores of stainless steel) either underivatised (A) or coupled with rabbit anti mouse immunoglobulin antibodies at 3 mg/ml (B and C). The particles were contacted with a Cy3-labeled purified monoclonal mouse antibody either in PBS (FIG. 4a) or spiked to whole heparinized bovine blood (FIG. 4b) in a batch incubation followed by wash in PBS. Particles were inspected for fluorescence at 570 nm (A+B) and also in normal light (C). The presence of Cy3-fluorescent molecules in the particles is revealed by a bright red emission as is seen for the antibody-coupled particles but to a much lesser degree for the non-derivatised particles with the Cy3-immunoglobulin in PBS as well as in blood.

FIG. 7 shows that after incubation of whole EDTA-stabilised human blood in a batch procedure with stainless steel/agarose-PEI particles some of the blood cells bind to the outer surface of the particles (A) while others are not bound (B) (FIG. 7A). On closer inspection (FIG. 7B, double arrow represents approximately 25 micrometer) the stainless steel core particle (A), the agarose coating layer (B) and unbound (C) as well as bound blood cells (D) are seen.

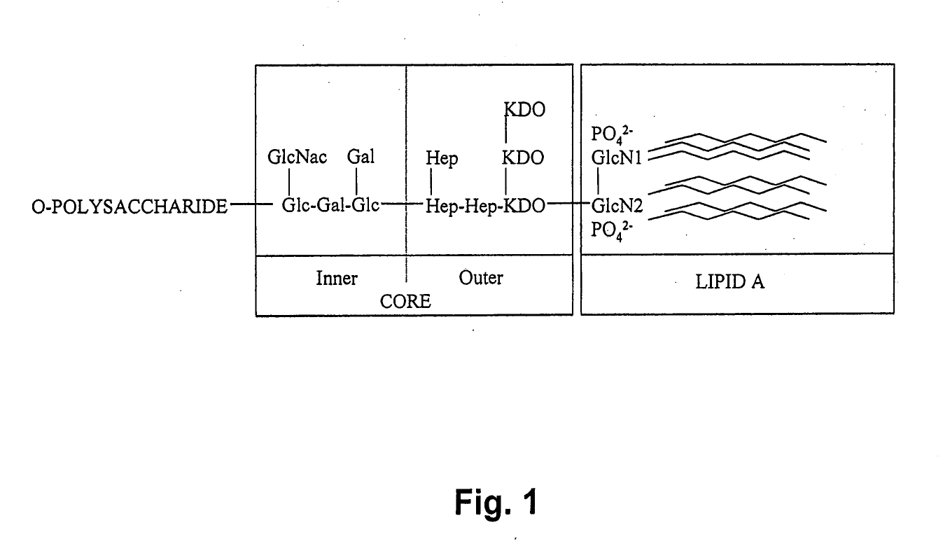

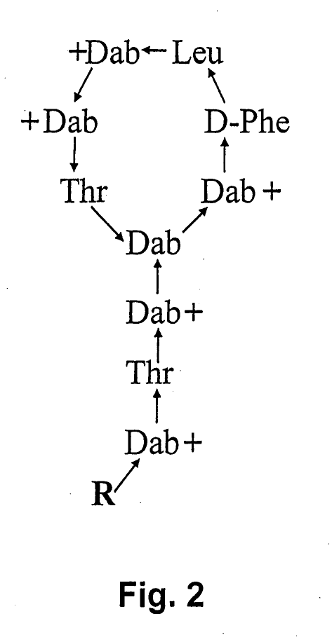

### DETAILED DESCRIPTION OF THE INVENTION

The present invention is based on the interesting finding that a particular stabilised fluidised bed adsorption column assembly has proven very useful for the preparation of a medical device in the treatment of sepsis caused by Gram-negative and possibly also Gram-positive bacteria in a mammal. The mammal is in particular a human being.

The adsorption column assembly comprises a column and an adsorption medium in the form of particles. In order for the adsorption column assembly to function properly as a stabilised fluidised bed adsorption column assembly, it is important that the sedimented volume of the particles is at the most 80% of the volume of the column. In most instances the sedimented volume of the particles is at the most 70% of the volume of the column, such as at the most 60% of the volume of the column, e.g. at the most 50% of the volume of the column. It is however envisaged that the sedimented volume of the particles should be at least 5% of the volume of the column. In some interesting instances, the sedimented volume is preferably 5-50%, such as 5-40%, e.g. 5-30%, of the volume of the column. The “sedimented volume of the particles” refers to the volume of the particles when present in pure water in a non-fluidised state. The volume can easily be measure by filling a suspension of the particles in water in a measuring flask.

The “volume of the column” refers to the total volume of the enclosure defined by the column. Due to the fact that columns often have fairly regular dimension, e.g. a cylindrical shape, the volume can easily be calculated. Alternatively, the column can be filled with water, and the volume of the water can subsequently be measured in a measuring flask.

It should be noted that the volume of the column should be measured/calculated when the column is arranged according to its intended use. Thus, the column assembly (column+particles) may further comprise a plunger which is arranged to compress or hold the particle in place when shipped.

A crucial part of the adsorption column assembly is the particles. The particles are characterised by carrying an affinity specific molecule with a specific affinity for the LPS portion of the
i) the LPS portion of the Gram-negative bacteria, and/or
ii) Gram-positive bacteria or harmful substances derived from said Gram-positive bacteria.

The term “harmful substance derived from said Gram-positive bacteria” means an entity promoting the development of sepsis and being either a constituent of Gram-positive bacteria or a secondary species to Gram-positive bacteria causing sepsis. Examples of such harmful substances are peptidoglycans, teichoic adds and exotoxins from Gram-positive bacteria, complexes of macromolecules with cells in the blood of the patient, said macromolecules including peptidoglycans, teichoic acids and exotoxins, as well as bacterial cells carrying peptidoglycans, teichoic acids and exotoxins on their surface.

In the currently most preferred embodiment, the affinity specific molecule have (at least) specific affinity for the LPS portion of the Gram-negative bacteria which cause sepsis.

It should be understood that the term “LPS portion of Gram-negative bacteria” covers the LPS in connection with the bacteria (possibly embedded in the membrane structure of said bacteria) as well as the LPS in free form.

The adsorption medium is typically a medium specially designed for use in an expanded bed processes, e.g. as illustrated in WO 00/57982, the disclosure of which is incorporated herein by reference.

The optimal density difference between the blood and the particles is obtained by providing particles having a very high density (e.g. significantly higher than the density of blood). Thus high-density particles will sink in the blood. However, it should also be mentioned that a stabilised fluidised bed can also be created, mutatis mutantis, by applying a downward flow of liquid to a bed of particles having densities and/or sizes allowing them to float in aqueous buffers. In this instance, the density should generally be the inverse of the below-stated limits and ranges.

Said adsorption medium typically has a density of 1.3-20 g/ml, such as at least 2.0, at least 3.0, at least 3.5 and preferably 4.0-16 g/ml.

In the present context the “density” of particles is the density of particles in the hydrated state.

It is believed that the relatively small diameter of the particles combined with the high density play an important role for the efficient stabilised fluidised bed processes. Thus, the average diameter of the particles of the adsorption medium is preferably 5-75 μm, such as in the range of 10-60 μm, such as in the range of 12-49 μm, more preferable in the range of 20-40 μm and even more preferable in the range of 10-30 μm.

Furthermore, it is believed that a relatively narrow particle size distribution is advantageous (bearing in mind that a certain width of the size distribution is advantageous when the material is to be use in a fluidised bed set-up). Thus, it is believed that at least 95% of the particles should have a diameter in the range of 5-80 μm, such as 15-45 μm, preferably in the range of 20-40 μm.

Said adsorption medium is typically in the form of particles having a density of at least 1.3 g/ml and a mean diameter in the range of 5-1000 μm, such as a density of at least 1.5 g/ml and a mean diameter in the range of 5-300 μm, preferably a density of at least 1.8 g/ml and a mean diameter in the range of 5-150 μm, and most preferred a density of more than 2.5 g/ml and a mean diameter in the range of 5-75 μm.

The high density is primarily obtained by inclusion of a high proportion of a dense core material, preferably having a density of at least 3.0 g/ml, such as at least 5.0, preferably in the range of 6.0-16.0 g/ml. This will result in particles which are pellicular or a conglomerate in composition. Examples of suitable core materials are inorganic compounds, metals, elementary non-metals, metal oxides, non-metal oxides, metal salts, metal alloys, and tungsten carbide, etc. as long as the density criterion above is fulfilled. It is preferred that the core material of at least 95% of the particles is a steel bead having a diameter in the range of 2-40, such as 8-28 μm, preferably 5-25 μm.

In another embodiment the core material of at least 95% of the particles is a tungsten carbide particle having a diameter in the range of 2-40, such as 15-38 μm, preferably 5-30 μm.

Furthermore, it is preferred that at least 95% of the particles comprises one core material having a diameter which is at least 0.70 time, such as at least 0.80 time or at least 0.85 time the diameter of the particle.

Alternatively, the core material is constituted by more than one bead, e.g. particles having a diameter of less that 10 μm.

Typically, the core material constitutes 10-99%, preferably 50-95%, of the volume of the particles, and the polymer base matrix constitutes 1-90%, preferably 5-50%, of the volume of the particle.

When the core material of a large proportion of the particles (>95%) is constituted by one bead, the polymeric base matrix is typically less than 50 μm in thickness. “Thickness” is defined as the geometrical distance between the core material and the surface of the particle. The thickness is preferable less than 20 μm, even more preferable less than 10 μm, and most preferable less than 5 μm in thickness. In one embodiment, it is envisaged that the polymeric base matrix may constitute a mono molecular layer covering the core material. Thus, in this instance, it is contemplated that the polymeric matrix may be replaced with low-molecular weight species having a predominant affinity for the core material. This affinity between the low-molecular species and the core material may be improved by surface treatment of the core material, e.g. by organosilylation of ceramic materials. The monomolecular layer may also be covalently coupled to the surface of the core material by chemical means as appreciated by those skilled in the arts of chemistry.

A very important feature of the adsorption medium is the fact that the particles on the polymeric base matrix carries an affinity specific molecule.

In the context of the present invention, the term “affinity specific molecules” is used to describe molecules that are characterised by their ability to associate specifically with the entity of interest (e.g. LPS portion of Gram-negative bacteria) under the conditions prevailing in blood.

It should be noted that more than one different affinity specific molecules may be present on particles within the adsorption medium. Thus, the adsorption medium may comprise different pools of particles each having different affinity specific molecules, and/or each particles may carry different affinity specific molecules.

The affinity specific molecule may for example be selected from the group consisting of immunoglobulins (including poly- and monoclonal antibodies), and sequence specific affinity specific molecules such as peptides, oligonucleotides, receptor proteins, including CD14 and comprising also Toll-like receptors and Toll-like receptor accessory proteins, antibiotics such as Polymyxin B, and lectins. These examples are well suited for adsorption of LPS portion of Gram-negative bacteria.

In one embodiment, the affinity specific molecules are selected from immunoglobulins.

In the currently most preferred embodiment, the affinity specific molecule is Polymyxin B.

In a preferred embodiment of the present invention the affinity specific molecule is selected from the group consisting of a Toll-like receptor, most preferably TLR4 or binding fragments thereof or multimeric arrangements thereof, CD14, MD2, TLR2 and LBP, and any combination thereof.

Such affinity specific molecules may be linked to the base matrix by methods known to the person skilled in the art, e.g. as described in “Immobilized Affinity Ligand Techniques” by Hermanson et al., Academic Press, Inc., San Diego, 1992, which is incorporated herein by reference. In cases where the polymeric base matrix do not have the properties to function as an active substance, the polymeric base matrix (or matrices where a mixture of polymers are used) may be derivatised (activated) to form a reactive substance that can react with functional chemical groups forming a chemical covalent bond under appropriate circumstances. Thus, materials comprising hydroxyl, amino, amide, carboxyl or thiol groups may be activated or derivatised using various activating chemicals, e.g. chemicals such as cyanogen bromide, divinyl sulfone, epichlorohydrin, bisepoxyranes, dibromopropanol, glutaric dialdehyde, carbodiimides, anhydrides, hydrazines, periodates, benzoquinones, triazines, tosylates, tresylates, and/or diazonium ions, in particular divinyl sulphone or epichlorohydrin linkers.

Immobilisation of antibodies to activated surfaces is well described elsewhere (see e.g. Harlow & Lane, 1988, Antibodies a Laboratory Manual, Cold Spring Harbor Laboratories) and comprises contacting the antibody molecules at specified conditions of pH, salinity and temperature with chemically activated particles for a specified length of time.

The polymeric base matrix is often used as a means of covering and keeping multiple core materials together and as a means for binding the affinity specific molecule. Thus, the polymeric base matrix is to be sought among certain types of natural or synthetic organic polymers, typically selected from
A) natural and synthetic polysaccharides and other carbohydrate based polymers, including agar, alginate, carrageenan, guar gum, gum arabic, gum ghatti, gum tragacanth, karaya gum, locust bean gum, xanthan gum, agaroses, celluloses, pectins, mucins, dextrans, starches, heparins, chitosans, hydroxy starches, hydroxypropyl starches, carboxymethyl starches, hydroxyethyl celluloses, hydroxypropyl celluloses, and carboxymethyl celluloses.
B) synthetic organic polymers and monomers resulting in polymers, including acrylic polymers, polyamides, polyimides, polyesters, polyethers, polymeric vinyl compounds, polyalkenes, and substituted derivatives thereof, as well as copolymers comprising more than one such polymer functionality, and substituted derivatives thereof; and
C) mixture thereof.

A preferred group of polymeric base matrices are polysaccharides such as agarose.

The ideal and preferred shape of a single particle is substantially spherical. The overall shape of the particles is, however, normally not extremely critical, thus, the particles can have other rounded shapes, e.g. ellipsoid, droplet and bean forms, as well as more irregular shapes.

In one preferred embodiment, the adsorption medium has a density at least 1.3, such as at least 2.0, preferably at least 3.0, more preferably at least 3.5, most preferred at least 4 g/ml, where the particles have an average diameter of 5-75 JAM, and the particles are essentially constructed of a polysaccharide base matrix and a core material.

In another embodiment, the adsorption medium has a density in the range of 6-16 g/ml, where the particles have an average diameter of 10-30 μm, and the particles are essentially constructed of a polysaccharide base matrix and a core material.

In a further embodiment, the adsorption medium has a density of at least 2.5 g/ml, where the particles have an average diameter of 5-75 μm, and the particles are essentially constructed of a polymeric base matrix selected from polysaccharides, preferably agarose, and a core material, said core material having a density in the range of 6.0-16.0 g/ml where at least 95% of the particles comprises one core material bead having a diameter which is at least 0270 of the diameter of the particle.

Alternatively, the adsorption medium may be in the form of conglomerate particles as disclosed in WO 92/18237 and WO 92/00799 or it may be any other type of particle having the desired characteristics in terms of e.g. size, density, surface chemistry, stability and safety. The particle may be either porous and permeable to the entity of interest or substantially non-porous and non-permeable having only the surface area available for binding of the entity of interest.

In the present context the expression “conglomerate” is intended to designate a particle of the adsorption medium, which comprises particles of core material of different types and sizes, held together by the polymeric base matrix, e.g. a particle consisting of two or more high density particles held together by surrounding agarose (polymeric base matrix) as described in WO 92/18237 and WO 92/00799. The expression “pellicular” is intended to designate a composite of particles, wherein each particle consists of only one high density core material coated with a layer of the polymeric base matrix, e.g. a high density stainless steel bead coated with agarose.

As mentioned above, the adsorption column assembly is adapted for fluidised bed adsorption, in particular stabilised fluidised bed adsorption.

A “fluidised bed” is herein defined as any arrangement of agitation, buffers and adsorbent particles in which a space between the individual particles wider than the minimum space obtained in a packed column of said particles is achieved. Thus, according to this definition, any set of particles that are utilised in any type of non-packed bed reactor constitutes a “fluidised bed”. Examples of such fluidised beds are fluidised beds obtained by applying fluid flow to an initially packed bed of particles at flow rates high enough to effect an expansion and “fluidisation” of the bed as described in chemical engineering textbooks (e.g. H. Scott Fogler in “Elements of Chemical Reaction Engineering”, p. 786, Prentice-Hall PTR, 1999).

A “stabilised fluidised bed” is defined as a fluidised bed in which there is a low degree of back-mixing of the adsorbent particles as a consequence of the movement of each particle being restricted to a limited volume of the total bed volume. This means that each particle has a low extent of axial dispersion and does not have the same probability of being found at any position within the confined space of the fluidised bed. A stabilised bed thus may be characterised as having a non-homogenous composition of the entire fluidised bed as the absence of back-mixing precludes mixing of mutually heterogenous zones of the bed. By the term “expanded bed” is meant a stabilised fluidised bed of particles created by applying an upward liquid flow of sample or of aqueous buffer through an inlet at the bottom of a column containing the bed of particles, said particles having a density and/or size distribution that make them position within a confined space of the fluidised bed. Like a stabilised fluidised bed, an expanded bed is characterised by having a low degree of back-mixing of the particles. With the exception of magnetically stabilised fluidised beds, the terms “expanded bed” and “stabilised fluidised bed” are to a large extent synonymous. In the present context, the terms “expanded bed adsorption” or “stabilised fluidised bed adsorption” describes the particular chromatographic technology wherein an adsorbent medium contained in a column having an inlet and an outlet is allowed to rise from its settled state by application of a fluid stream of e.g. the sample (body fluid) or an aqueous buffer in an upward flow, thereby increasing the space between the particles. This can happen simultaneously with or prior to the introduction of the fluid sample.

The fact that, in a stabilised fluidised bed, blood is passed through a bed consisting of adsorption particles substantially without turbulence and back-mixing provides an efficient and gentle contact with a large surface area of an adsorption medium ensuring that the majority of the entities of interest which are to be separated from the extracellular body fluid are detained. It is well known from packed bed column plug flow that elimination of turbulence, back-mixing and channel formation provide a very efficient adsorption method due to the high number of theoretical plates formed.

The number of “theoretical plates” In a chromatographic system is an expression of the number of equilibria that can be formed between the particles of the adsorption medium and the sample component interacting with the bed of particles. This number is expressed in number per meter column and can be calculated from the residence time of a suitable tracer being pumped through the column as known to a person skilled in the art, see e.g.
The Amersham Biosciences booklet “Expanded Bed Adsorption Handbook, Ref. no. 18112426”, which is available at http://www.chromatography.apbiotech.com, and furthermore is incorporated herein by reference.

Typically, the flow rate of the blood through the column assembly is such that expansion ratio (ratio of height of adsorption medium in expanded state to height of the adsorption medium in sedimented stated) of the fluidised bed is at least 1.3, such as at least 1.5. In may instances (which often are preferred), the flow rate is however such that the expansion ratio is at least 2.5 such as at least 4.0 or event at least 5.0.

The flow rate of the blood is preferably adjusted in such a manner that a stabilised fluidised bed of the particles is formed.

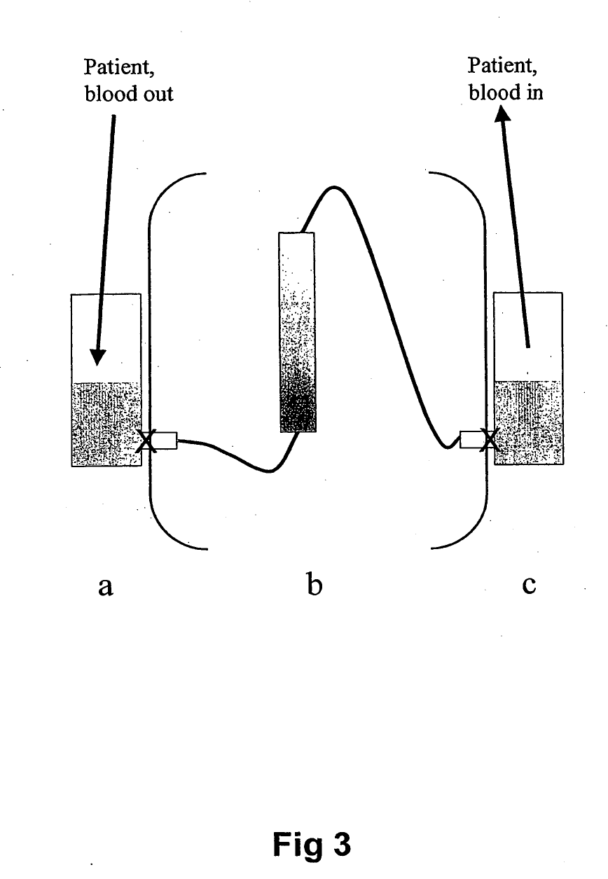

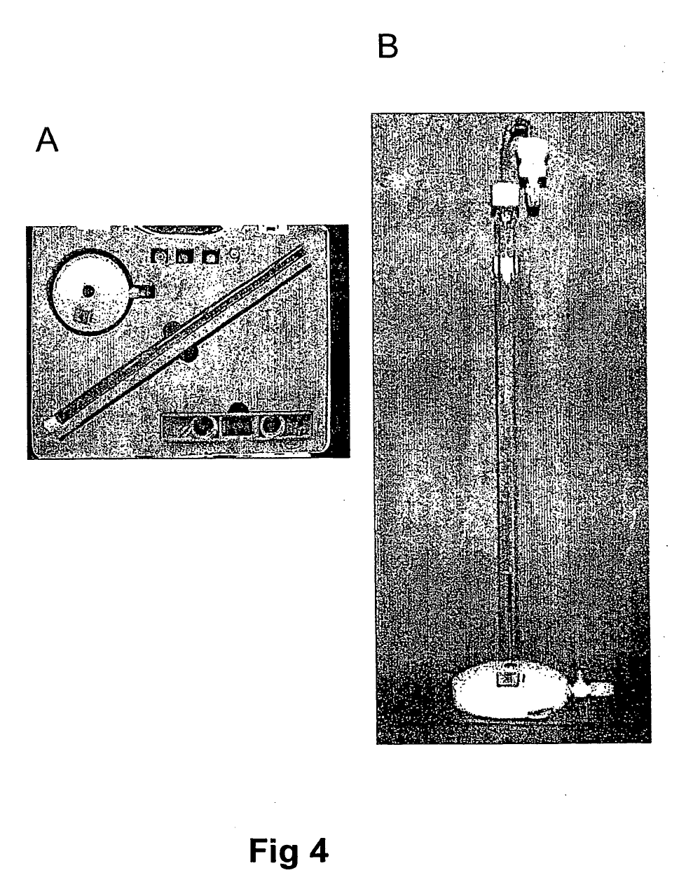

The adsorption column assembly typically further comprises inlet means and outlet means. When used, the column is places in an upright position (longitudinal axis of column cylinder in a vertical direction). Thus, the bottom part will hold the inlet means. Further, the bottom part may have a bottom part means to prevent the particles from entering the inlet means. See FIG. 3. In some embodiments, the adsorption column assembly includes means for agitation, e.g. a propeller, a magnetic bar for magnetic stirring, or the like, so as to ensure a uniform distribution of the blood into the column.

By the term “magnetically stabilised fluidised bed” is meant a stabilised fluidised bed of adsorbent magnetizable particles obtained by placing particles in a radially uniform magnetic field parallel to the path of fluid flow through the bed.

The adsorption column assembly is useful for the preparation of a medical device for the treatment of sepsis in a mammal by extracorporeal adsorption. The medical device may beside the adsorption column assembly include valves, pumps, tubes, container reservoirs, etc. An sketch of a medical device is presented in FIG. 3.

The blood from the patient (mammal), preferably whole blood, can be led to a container reservoir where a sufficient portion is collected before the otherwise continuous extracorporeal adsorption process is initiated. After passage of the adsorption column assembly, the treated blood will also be collected in a container reservoir so as to allow the treated blood to passively revert to the mammal (typically a human being) under treatment. It should be understood that valves, pumps, tubes, container reservoirs, etc. normally used in handling mammalian blood can be used in connection with the medical device of the invention.

By “continuous process” Is meant a process that can be defined by a constant function being applied at the starting time point of the process and terminated at the end point of the process. Thus, in the present context a typical example of a continuous process is a procedure in which whole blood is removed at a constant flow (i.e. substantially uninterrupted flow) from a patient (a mammal) and also reintroduced into the patient with a constant flow. As the flow rate of the whole blood from the patient does not necessarily fit with the optimal flow rate of the whole blood through the adsorption column assembly or with the possible flow rate when reinfusing the treated blood into the same patient (mammal), a container reservoir is suitably arranged upstream relative to the adsorption column assembly, and another container reservoir is suitably arranged downstream relative to the adsorption column assembly.

In other words it is to be understood that the removal of blood from the patient (the mammal) at a given flow rate (step a)), the process of contacting of the blood with the adsorption medium (step b)) and the reinfusion of the blood into the patient (step c)) is performed in one consecutive and interrelated procedure at the patient's bed site.

This procedure is to be understood in contrast to any other “discontinuous” procedure wherein the body fluid is withdrawn from the patient in one independent procedure at one time, optionally stored and contacted with an adsorption medium in a batch-wise manner at another time and reintroduced into the patient at still another time chosen largely independent of the two first procedures.

The best mode for performing extracorporeal adsorption with a human patient is well known to those skilled in the art and typically comprises creating a venovenous shunt with flow rates in the 100 to 200 ml/min range and applying a suitable anticoagulant as e.g. citrate or heparin at concentrations keeping the activated clotting time between 160-180 s.

At this concentration of anticoagulant it is not necessary to remove it before the blood is reintroduced into the patient.

In one embodiment, the steps (a), (b) and (c) are preceded by a initial step by which a substance is first injected into the blood stream of the mammal.

In another embodiment, the continuous procedure consisting of steps (a), (b) and (c) is initiated upon the activation of a switch directing the blood from the patient to the stabilised fluidised adsorption medium and back into the patient. In this embodiment the switch is put in line with the patient's blood circulation, allowing the passage of the blood into the patient again in its resting state; it is connected with a continuous blood monitoring device, capable of activating the switch when the monitoring device records a pre-set change in the plasma concentration of a suitable biomarker, e.g. an acute phase protein like C-reactive protein or serum amyloid A or any other substance reacting to initiation of a sepsis-like condition. By this method a continuous, unattended surveillance of a patient at risk for developing sepsis can be achieved, the method reacting to sepsis-like changes in blood parameters by shunting the blood through the stabilised fluidised adsorption device of the invention (stand-by extracorporeal adsorption).

In a preferred embodiment the particles making up the stabilised expanded bed of the present invention are very small (mean diameter of 15-20 μm) ensuring af very high surface area per volume packed bed and allowing the use of non-porous particles for adsorption of the entities of interest on the surface of the particles with sufficiently high capacity.

However, the present invention is characterised by the ability to bind soluble harmful substances as well as suspended harmful substances (cells) which constitutes a major advantage to the other methods hitherto known in the art as described above. A method employing the stabilised fluidised bed in an extracorporeal adsorption process for the treatment (therapeutic and profylactic treatment) of sepsis is thus greatly improved compared to other such methods, as, in addition to binding and depleting the blood of soluble, harmful substances, the specific bio-macromolecular entitles being bound by the affinity-specific molecules of the fluidised bed will also be bound when present on cells in the blood stream and furthermore will be able to bind cells from the blood, provided that said cells can also bind the biomacromolecular entity.

One preferred example of the use of the method in which advantage is derived from the binding interactions described above is the use of an expanded bed containing immobilised lipolysaccharide-binding substances on the particles of the expanded bed. When patient's blood is circulated through this expanded bed, soluble lipopolysaccharlde will be bound to the particles of the bed. However, also lipopolysaccharide present on the surface of bacterial cells or fragments thereof and lipopolysaccharide bound by the patient's own cells (for example CD14 positive monocytic cells) and circulating as such in the blood stream of the patient will be allowed to bind to the particles of the bed, effecting their removal from the blood stream. Furthermore, in addition to this, LPS-moieties bound to the particles of the expanded bed will act as immobilised affinity-specific molecules being able to interact with cells of the blood stream from the patient, such cells being typically CD14- and TLR4 positive monocytes which are known to bind lipopolysaccharides. This greatly expands the clean-up of harmful substances from the blood leading to the removal of all LPS-related and LPS-binding material from the blood stream, facilitating and speeding up the treatment process.

It should be stressed that the extracorporeal adsorption priniciple allows the use of antibodies and other substances having affinity for the harmful substance being removed from the blood, even if these substances have unknown or non-beneficial pharmacokinetics when injected, are toxic to the patient, and/or have non-adequate affinities for in vivo binding of the harmful substance in question; this broadens the range of substances that will be useful for creating an adsorptive medium for said purpose.

The method described herein is expected to result in a reduction of LPS by at least 80% in 60 minutes while recoveries of other substances in the blood, in particular non-related serum proteins are expected to be above 85%.

The term “sepsis” Is used here as synonymous with septicaemia, that is the presence of bacteria in the blood circulatory system.

### EXAMPLES

### Example 1

### The Basics of a Stabilised Fluidised Bed Procedure

A. Running Human Whole Blood Through a Stabilised Fluidised Bed (EDTA-Stabilised Blood)

The purpose of the following example is to demonstrate the feasibility of running human non-separated blood through a stabilised fluid bed of high density, low diameter adsorbent particles.

Materials and Methods:

The experimental fluidised bed column set-up was established based on the following standard laboratory equipment:
Pump (Ole Dich Aps, Denmark)
Silicone tubing (MasterFlex)
Magnetic stirrer (Janke and Kunkel)
Column: UpFront Chromatography A/S, Denmark (cat. no. 7010-0000), diameter 1.0 cm, height 50 cm.
   Adsorbent Particles (without Ligand):

Test-particles were provided by UpFront Chromatography A/S, Denmark. The particles had the following characteristics:
Bead composition: epichlorohydrin cross-linked agarose (4% w/v) with a core of tungsten carbide
Bead shape: Mainly spherical
Diameter: 20-40 μm
Average individual bead density in the hydrated state: 4.1 g/ml
Void volume in sedimented state: Approx. 40% of packed volume
Theoretical bead surface area per litre sedimented particles: Approx. 120 m²
   (theoretical surface area was calculated from estimating that 1 litre sedimented bed corresponds to 600 ml particles (the non-void volume), each having a volume of 14,130 μm³ from which the number of particles could be calculated to be 42×10⁹. With each bead having an outer surface area of 2826 μm² this gives a total bed surface area of 120 m² for 1 litre of particles).
   Adsorbent Equilibration Buffer:

6% w/v dextran MW 110.000 (Pharmacosmos, Denmark) in 0.9% w/v sodium chloride was used to pre-equilibrate the adsorbent before percolation of the blood through the column.

Blood:

A freshly drawn human blood sample from a healthy donor, collected in standard EDTA glass tubes (Becton Dickinson, code no. 15067), was used for the experiment. The blood was stored at room temperature and used within 1 hour after collection.

Procedure:

The fluid bed column (diameter: 1 cm) was assembled according to the supplier's instructions and added to an aqueous suspension of the adsorbent particles to reach a sedimented bed height of 7 (5.5 ml, corresponding to approx. 0.7 m² bead surface area). Then an upward flow of the adsorbent equilibration buffer of approx. 5 m/min was applied in order to fluidise and wash the particles with the buffer and in order to ensure an optimal salt concentration/osmolality for minimal hemolysis of the blood cells when entering the column.

The column was adjusted to a completely vertical position in order to secure an even flow inside the column.

When the particles were fluidised (i.e. when the fluidised bed height reached above 10 cm) the magnetic stirrer at the bottom of the column was engaged at approx. 80% full speed in order to ensure an even distribution of the incoming liquid and the flow rate was adjusted to 2.2 ml/min. The washing with adsorbent equilibration buffer was continued for 15 min. In which time a stabilised fluidised bed was formed with a fluidised bed height of 16 cm. The stability of the fluidised bed was ascertained by a careful visual inspection of the bed and the particles inside the column using a magnifying glass as a visualisation aid. The lack of visual channelling and back mixing was taken as an indication of the bed stability.

Following the establishment of a stabilised and equilibrated fluid bed, 100 ml human blood was pumped into the column at a steady flow rate of 2.2 ml/min.

Results:

The entry and penetration of the blood into the stabilised fluidised bed was carefully followed by visual inspection: A well-defined weakly parabolic front of the red blood sample was moving at constant speed through the stabilised fluidised bed and no back-mixing and channelling was observed anywhere in the entire stabilised fluidised bed. When the blood sample occupied the entire volume of the stabilised fluidised bed the height of the bed had increased to 21 cm (i.e. 3 times the sedimented bed height). Although the non-transparency of the blood sample made it very difficult to observe the adsorbent particles inside the column, the use of a magnifying glass made it possible to ascertain the bed height as well as the absence of channelling in the bed.

Following the break-through of the blood sample at the top of the stabilised fluidised bed, the run through was collected in fractions of 5 ml blood while continuing the application of the full 100 ml blood sample to the column. The collected fractions were centrifuged at 500 G for 10 min and the degree of hemolysis occurring after passage of the sample through the column was determined by spectrophotometry at 540 nm using an untreated blood sample as a reference sample. All collected fractions had a degree of hemolysis below 2% of the total number of erythrocytes (as determined in a fully experimentally hemolysed control sample). Further, microscopic examination of the collected blood samples did not reveal any occurrence of clotting of the blood and neither could any adsorbent particles be detected in the samples.

Following the application of the 100 ml blood sample, the column was percolated with the adsorbent equilibration buffer again with the aim of washing out the remaining blood inside the column. The washing was also conducted at a flow rate of 2.2 m/min. The entry of the buffer and the gradual washing of the column gave rise to a sharp and upwardly moving boundary between the incoming equilibration buffer and the blood sample, indicating a stable fluidisation of the bed, devoid of channelling and back mixing, with fluids moving in plug flow. When the blood sample had been completely washed out of the column, the fluidised bed height had returned to 16 cm.

Following the washing of the stabilised fluidised bed with equilibration buffer a sample of the particles from inside the column was microscopically examined. It was observed that all particles were fully intact with smooth surfaces and no cells adhering to them. In this experiment an EDTA-stabilised, human full blood sample of 100 ml was applied to and pumped through a stabilised fluidised bed with a sedimented bed height of 7 cm (5.5 ml). The overall conclusion is that although it could be expected to give rise to significant problems to apply full blood comprising 40-50% by volume of blood cells as well as a high concentration of proteins to a stabilised fluidised bed of small adsorbent particles in the form of the break-down of the stabilised fluid bed system and/or significant negative effects on the blood, no serious problems were observed during the entire procedure. Furthermore, particles could be washed completely free of blood and the entire bed was reversed to its initial state after passage of the blood and subsequent washing.

B. Running Human Whole Blood Through a Stabilised Fluidised Bed (Heparin-Stabilised Blood)

The same experiment as in Example 1A was performed with the only exception that heparinised human blood was used instead of EDTA stabilised blood. The blood for this experiment was collected in standard heparin glass tubes (Venoject, NaHeparin, Terumo Europe).

The results obtained were similar to the results described above for EDTA-stabilised human blood and thus indicate that the stabilised fluidised bed procedure can be performed without problems with heparinised human blood as well as with EDTA stabilised blood.

### Example 2

### Specific Adsorption of an Enzyme-Conjugate from Whole Human Blood in a Stabilised Fluidised Bed; Binding of Avidin-Peroxidase by Biotin-Coupled Particles

The aim of the following experiment was to establish the feasibility of binding of a specific bio-macromolecular entity of interest from whole human blood in a stabilised fluidised bed procedure. For the sole purpose of demonstrating such binding, an enzyme-conjugate was used as a model protein as this allowed a sensitive assay to be performed in order to demonstrate the binding of the enzyme. The test substance (peroxidase-labelled avidin) was added to whole human blood followed by adsorption of the test substance to a high-density biotin labelled adsorbent in a stabilised fluidised bed procedure. Binding of the test substance to the adsorbent was then demonstrated by the development of staining on the adsorbent particles though the action of the bound peroxidase conjugate using a suitable indicator enzyme substrate (diaminobenzidine).

This example supplements Example 1 in showing the feasibility of using another type of particles with a lower density and bigger diameter and with a core of glass particles for stabilised fluidised bed chromatography of whole blood.

Materials and Methods:

The experimental set-up and equipment used was the same as in Example 1.

Adsorbent Particles (with Biotin as Ligand):

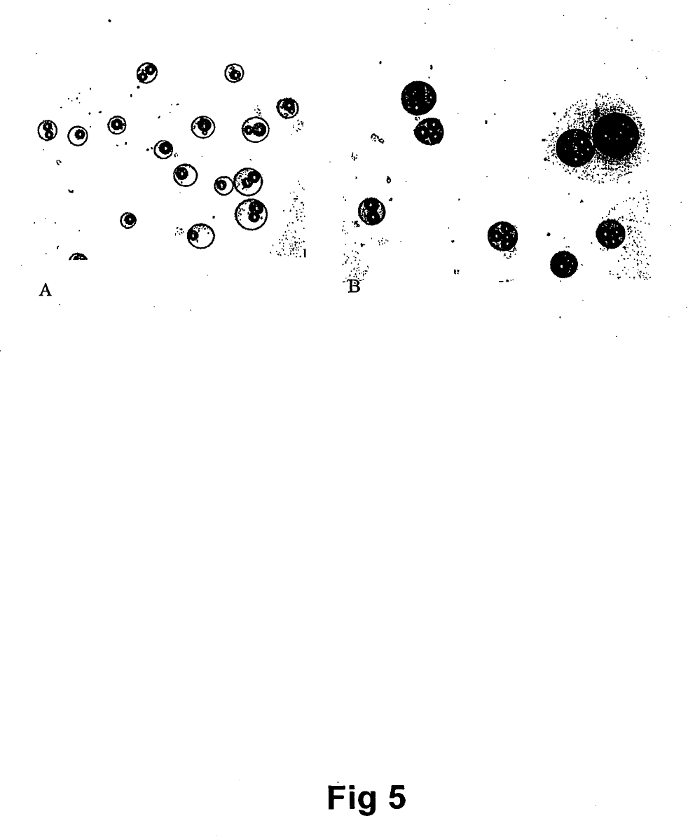

The adsorbent used for this experiment was a high-density biotin-agarose/glass adsorbent (product no.: 6302-0000, UpFront Chromatography A/S, Denmark). This adsorbent has the following characteristics:
Bead composition: epichlorohydrin cross-linked agarose (6% w/v) with a core of spherical glass particles (See also FIG. 5)
Bead shape: Mainly spherical
Diameter: 100-300 μm
Average individual bead density in the hydrated state: 1.5 g/ml
Ligand: Biotin.
   Adsorbent Equilibration Buffer:

6% w/v dextran MW 110.000 (Pharmacosmos, Denmark) in 0.9% w/v sodium chloride was used to pre-equilibrate the adsorbent before percolation of the blood through the column.

Blood:

A freshly drawn human blood sample from a healthy donor, collected in standard EDTA glass tubes (Becton Dickinson, code no. 15067), was used for the experiment. The blood was stored at room temperature and used within 1 hour after collection. Just prior to the adsorption procedure, 1 ml horseradish peroxidase labelled avidin (avidin-peroxidase, 1 mg/ml, Product no.: 4030Y, Kem-En-Tec A/S, Denmark) was added to 100 ml of the blood to give a final concentration of 10 μg avidin-peroxidase per ml human blood.

Procedure:

The fluid bed column was assembled according to the supplier's instructions and added an aqueous suspension of the adsorbent particles to reach a sedimented bed height of 5.8 cm.

In order to ensure an optimal salt concentration/osmolality for minimal hemolysis of the blood cells when entering the column, a wash with adsorbent equilibration buffer was initially performed at a flow rate of 2.2 ml/min. When the adsorbent particles were fluidised by the upward flow of equilibration buffer the magnetic stirrer was engaged at 80% full speed and the column was positioned carefully to a completely vertical state. When reaching a fully stabilised fluidised state, the height of the adsorbent bed had increased to 10.5 cm.

Following the initial wash with equilibration buffer, the blood sample was applied to the column with a flow rate of 2.2 ml/min. A well-defined weakly parabolic front of blood was then observed moving gradually up through the stabilised fluidised bed. No back mixing or channelling was observed throughout the experiment. When fully loaded with the human blood, the bed height was determined using a magnifying glass to be approx. 14.5 cm (i.e. 2.5 times the sedimented bed height).

The degree of hemolysis of the blood having passed the column was determined by spectrophotometry at 540 nm (as in Example 1) to be below 0.2%.

Following the application of the blood sample the column was washed with 200 ml adsorbent equilibration buffer in order to wash out the blood and any unbound peroxidase labelled avidin.

After washing the stabilised fluidised bed thoroughly, a sample of the adsorbent particles was incubated for 2 minutes with diaminobenzidine substrate (cat. no.: 4150, Kem-En-Tec A/S, Denmark) prepared according to the suppliers instructions.

Results:

The diaminobenzidine enzyme substrate gave a very strong brown colouring of the adsorbent particles thus demonstrating the presence on the particles of bound avidin-peroxidase extracted from the blood during the passage of the blood through the stabilised fluidised bed (see FIG. 5).

An experiment performed with the same type of adsorbent particles but lacking the biotin ligand gave a negative result in this enzyme substrate test thus indicating that the first result was due to a specific interaction and binding between the biotin ligand on the adsorbent particles and the avidin-peroxidase added as a test substance to the human blood sample (see FIG. 5)

### Example 3

### Specific Adsorption of Mouse Antibodies from Whole Bovine Blood in a Batch Operation, Binding of Mouse Immunoglobulin by Anti-Mouse Antibody-Coupled Particles

The aim of this example was to demonstrate the feasibility of using an anti-mouse immunoglobulin antibody-coupled adsorbent for the extraction of mouse antibodies added to whole bovine blood.

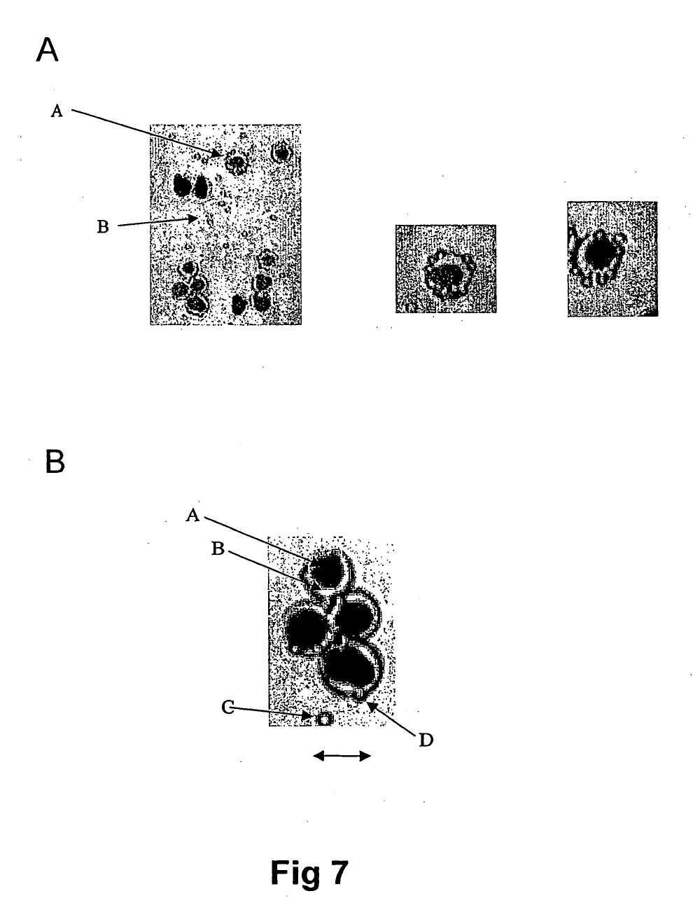

The adsorbent used for this experiment was a high density divinylsulfone-coupled agarose/stainless steel adsorbent (Upfront Chromatography A/S, Denmark) to which an anti-mouse immunoglobulin antibody from rabbits (code no. Z0109, DAKO A/S, Denmark) was coupled.
Bead composition: epichlorohydrin cross-linked agarose (4% w/v) with a core of stainless steel particles (FIG. 7)
Bead shape: Mainly spherical
Diameter: 20-40 μm
Average individual bead density in the hydrated state: 3.8 g/ml
Ligand: Rabbit anti-mouse immunoglobulin (DAKO A/S, Denmark, code no. Z0109) coupled through divinylsulfone at 3 mg/ml
   Procedure:

Freshly obtained heparinised whole bovine blood was obtained from a healthy donor collecting the blood in heparin-tubes (Venoject, NaHeparin, Terumo Europe). For the purpose of demonstrating the selective extraction of mouse immunoglobulin from the blood, 100 μl Cy3-labelled mouse immunoglobulin prepared from a kit obtained from Amersham Pharmacia Biotech (code no. PA33000) and according to the manufacturers instructions was added to 0.5 ml bovine blood and incubated (slow rotation) with a 100 μl suspension of adsorbent particles for 30 minutes at room temperature. The mouse immunoglobulin was protein A-purified, IgG1 isotype and used at 3.7 mg/ml in PBS. In parallel with this a similar incubation was performed with 100 μl Cy3-mouse immunoglobulin in PBS (no blood, positive control) and both of these incubations were also performed with non-coupled particles (negative control).

The adsorbent particles were washed prior to use by incubation and decanting with PBS (2 times) prior to the incubation with the blood/mouse antibody mixture. After 30 minutes of incubation, particles were retrieved by decantation, washed two times with PBS (incubation/decantation) and then inspected by visual and fluorescence microscopy (at 570 nm).

Results:

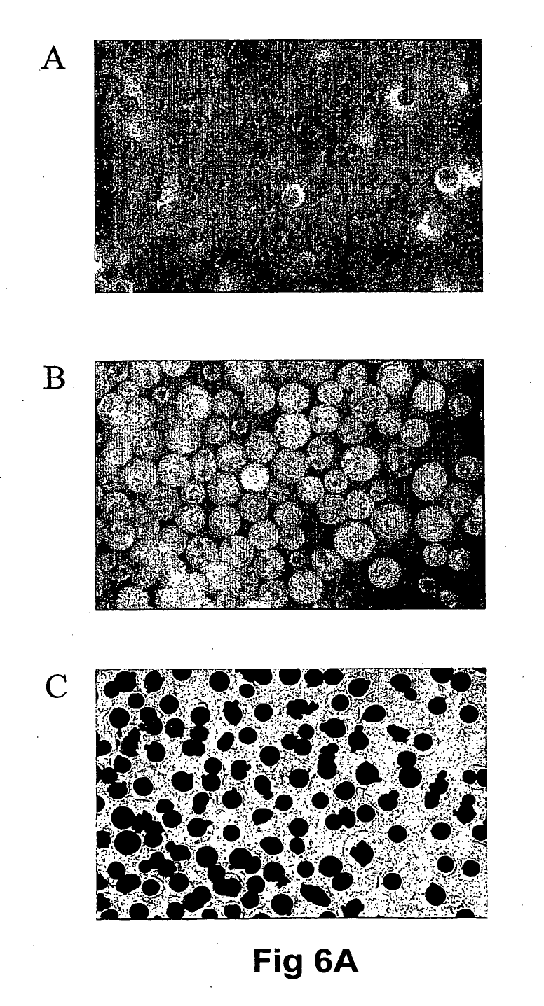

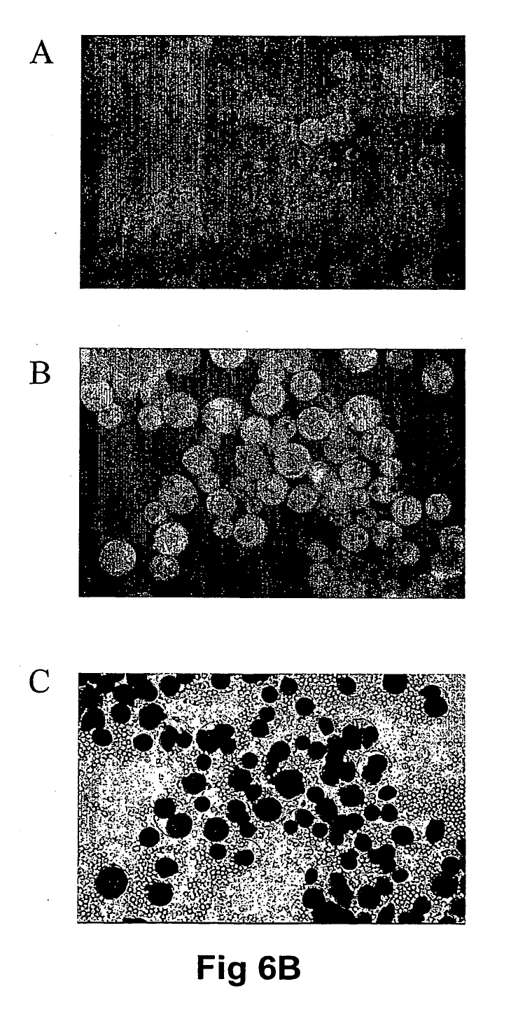

As seen in FIG. 6, the Z0109-derivatised particles bound the Cy-3-labelled mouse antibody both when supplied in pure solution (PBS) and spiked into whole heparinised blood at a 5 times lower concentration while a very low background binding was observed with non-derivatised particles. As the intensity of the fluorescence of the particles were similar when binding was performed with the pure Cy3-immunoglobulin solution as compared to when the incubation was performed with Cy3-immunoglobulin spiked to whole blood the binding of the immunoglobulin to the particles were clearly not affected by the presence of whole blood. Fluorescence was confined to the outer surface of the polymeric base matrix (the agarose layer) as would be expected (see FIG. 6).

In conclusion this experiment shows the feasibility of using small adsorbent particles for batch-wise specific retrieval of labelled immunoglobulin molecules from whole bovine blood.

### Example 4

### Adsorption of Blood Cells to a High Density Adsorbent in a Continuous Stirred Tank Reactor Using Polyethyleneimine-Coupled Particles

The aim of the following example was to demonstrate the feasibility of binding human blood cells to a high-density ion-exchange adsorbent in a stirred tank reactor. The adsorbent used for this experiment was a high-density polyethyleneimine (PEI) agarose/stainless steel adsorbent (UpFront Chromatography A/S, Denmark). This adsorbent has the following characteristics:
Bead composition: epichlorohydrin cross-linked agarose (4% w/v) with a core of stainless steel particles (See also FIG. 7).
Bead shape: Mainly spherical.
Diameter: 20-40 μm.
Average individual bead density in the hydrated state: 3.8 g/ml.
Ligand: polyethyleneimine (PEI)

Whole EDTA-stabilised human blood (100 ml) obtained as described in Example 1 was mixed with 1 ml adsorbent particles for 10 minutes under careful agitation at room temperature. Following sedimentation of the adsorbent particles, the blood sample was decanted and the particles were washed with adsorbent equilibration buffer (incubating and decanting the buffer) followed by microscopic examination.

As illustrated in FIG. 7 the adsorbent binds the blood cells to its surface adsorbent polymeric matrix layer.

### Example 5

### Specific Binding of Cells in a Cell Suspension Directly to Antibody-Coated High Density Conglomerate Particles in a Batch Process

The purpose of this example is to demonstrate that antibody-coated conglomerate adsorbent particles is useful for immuno-affinity chromatography of whole cells. The adsorbent used for this experiment is an divinylsulfone-activated (low activation level) agarose/stainless steel adsorbent (Upfront Chromatography A/S, Denmark) to which a mouse anti-bovine CD8 antibody (monoclonal, IgG1, ATCC CLR1871) is coupled:
Bead composition: epichlorohydrin cross-linked agarose (4% w/v) with a core of stainless steel particles (See also FIG. 7)
Bead shape: Mainly spherical
Diameter: 20-40 μm
Average individual bead density in the hydrated state: 3.8 g/ml
Ligand: Monoclonal mouse anti-bovine CD8 Immunoglobulin (ATCC CLR1871) coupled through divinylsulfone
   Procedure:

Freshly obtained heparinised whole bovine blood is obtained from a healthy donor by collecting the blood in heparin-tubes (Venoject, NaHeparin, Terumo Europe). Peripheral blood mononuclear cells (PBMCs) are prepared by standard methods (density gradient centrifugation through Ficoll™, (Amersham Pharmacia Biotech, code no. 17-1440-03) according to standard procedures (Rickwood ed., 1984, Centrifugation: a practical approach, IRL Press) and resuspended in PBS at approximately 106 PBMCs pr ml. This cell suspension is prepared from fresh blood and it is used immediately after preparation.

As a model experiment solely to demonstrate the ability of the adsorbent particles to bind whole cells efficiently, 500 μl of the PBMC suspension is first mixed with 100 μl Cy-3-conjugated antibody against bovine CD8 (the same antibody used for coupling to the particles) (3.7 mg/ml) prepared using a Cy3 labelling kit from Amersham Pharmacia Biotech (code no. PA 33000) according to the instructions supplied with the kit. After 30 minutes at room temperature this mixture is then incubated with 200 μl antibody-coupled particles in PBS. As a negative control, non-derivatised particles of a similar composition are also incubated in a separate experiment. The adsorbent particles are washed prior to incubations by incubation and decanting with PBS (3 times). After 30 minutes of gentle agitation at room temperature followed by 3 times wash in PBS, the resulting suspension is investigated in a fluorescence microscope in visual light and at 570 nm to reveal the presence and localisation of CD8-positive (Cy3-labelled) cells in the suspension.

Results:

It is to be expected from this experiment that the antibody-derivatised particles clearly show up under the microscope with the smaller CD-8-positive (and therefore Cy-3-fluorescent) PBMCs attached to the surface of the particles with none or very few non-fluorescent cells attached. This pattern is clearly different from the random pattern of fluorescent (CD8-positive) and non-fluorescent cells seen with non-derivatised conglomerate particles, further demonstrating the specific nature of the binding of cells to the immunoadsorbent particles.

### Example 6

### Specific Binding of Cells in a Cell Suspension Indirectly to Antibody-Coated High-Density Conglomerate Particles by Means of a Catching Antibody in a Batch Process

The purpose of this example is to demonstrate that antibody-coated conglomerate adsorbent particles can be used for indirect immuno-affinity chromatography of whole cells.

The adsorbent used for this experiment is a divinylsulfone-activated (low activation level) agarose/stainless steel adsorbent (Upfront Chromatography A/S, Denmark) to which a rabbit anti mouse immunoglobulin (DAKO code no. Z0109) is coupled:
Bead composition: epichlorohydrin cross-linked agarose (4% w/v) with a core of stainless steel particles (See also FIG. 7)
Bead shape: Mainly spherical
Diameter: 20-40 μm
Average individual bead density in the hydrated state: 3.8 g/ml
Ligand: Rabbit anti mouse immunoglobulin (DAKO Z0109 coupled through divinylsulfone
   Procedure:

Freshly obtained heparinised whole bovine blood is obtained from a healthy donor by collecting the blood in heparin-tubes (Venoject, NaHeparin, Terumo Europe). Peripheral blood mononuclear cells (PBMCs) are prepared by standard methods (centrifugation through Ficoll™, (Amersham Pharmacia Biotech, code no. 17-1440-03) according to standard procedures (Rickwood ed., 1984, Centrifugation: a practical approach, IRL Press) and resuspended in PBS at approximately 106 PBMCs pr ml. This cell suspension is prepared from fresh blood and it is used immediately after preparation.

As a model experiment solely to demonstrate the ability of the adsorbent particles to bind whole cells efficiently, 500 μl of the PBMC suspension is first mixed with 100 PI Cy-3-conjugated antibody against bovine CD8 (3.7 mg/ml) prepared using a Cy-3 labelling kit from Amersham Pharmacia Biotech (PA 33000) according to the instructions supplied with the kit. After 30 minutes at room temperature this mixture is then washed carefully 3 times to remove surplus of Cy3-labelled antibody and then incubated with 200 μl Z0109-coupled particles in PBS. As a negative control, non-derivatised particles of a similar composition are also incubated in a separate experiment. The adsorbent particles are washed by incubation and decanting with PBS (3 times) prior to the incubation with the PBMC. After 30 minutes of gentle agitation at room temperature followed by 3 times wash in PBS the resulting suspension is investigated in a fluorescence microscope in visual light and at 570 nm to reveal the presence and localisation of CD8-positive (Cy3-labelled) cells in the suspension.

Results:

It is to be expected from this experiment that the antibody-derivatised particles clearly show up under the microscope with the smaller CD-8-positive (and therefore Cy-3-fluorescent) PBMCs attached to the surface and none or very few non-fluorescent cells attached. This pattern would clearly be different from the random pattern of fluorescent (CD8-positive) and non-fluorescent cells seen with non-derivatised conglomerate particles, and would demonstrate the specific nature of the binding of cells to the immunoadsorbent particles.

Thus this experiment is designed to show that it is possible to prepare a “universal” immunoadsorbent for monoclonal antibodies of any kind, using a polyclonal mouse immunoglobulin-specific antibody as a ligand coupled to the adsorbent conglomerate particles. This is an advantage, as some monoclonal antibodies as known to a person skilled in the art will not function after covalent (chemical) immobilisation to solid surfaces. Furthermore this will allow the use of a stabilised fluidised bed generated from such general immunoadsorbent particles in a device for catching other antibodies, e.g. after the reaction in the solution phase of these second antibodies with constituents in body fluids (“bind-and-catch” approach).

### Example 7

### The Use of a Stabilised Fluidised Bed Comprising Immunoadsorbent High Density Conglomerate Particles for the Removal of CD8-Positive T-Cells in a Live Host

To demonstrate the feasibility of using a stabilised fluidised bed for the extracorporeal specific removal of a T-cell subset from the blood-stream of a cow, high density adsorbent particles are derivatised with an antibody against cow CD8 by coupling a monoclonal mouse antibody against bovine CD8 (ATCC CLR1871) through divinylsulfone to the particles.

Adsorbent Particles (without Ligand):

Test-particles are provided by UpFront Chromatography A/S, Denmark. The particles have the following characteristics:
Bead composition: epichlorohydrin cross-linked agarose (4% w/v) with a core of tungsten carbide
Bead shape: Mainly spherical
Diameter: 20-40 μm
Average individual bead density in the hydrated state: 4.1 g/ml
Void volume in sedimented state: Approx. 40% of packed volume
Theoretical bead surface area per litre sedimented particles: Approx. 120 m²
   Adsorbent Equilibration Buffer:

6% w/v dextran MW 110.000 (Pharmacosmos, Denmark) in 0.9% w/v sodium chloride is used to pre-equilibrate the adsorbent before percolation of the blood through the column.

Procedure:

The fluid bed column (diameter: 1 cm) is assembled according to the suppliers instructions and added an aqueous suspension of the adsorbent particles to reach a sedimented bed height of 7 cm (5.5 ml, corresponding to approx. 0.7 m² bead surface area). Then an upward flow of the adsorbent equilibration buffer of approx. 5 ml/min is applied in order to fluidise and wash the particles with the buffer and in order to ensure an optimal salt concentration/osmolality for minimal hemolysis of the blood cells when entering the column. The column is adjusted to a completely vertical position in order to secure an even flow inside the column. When the particles are fluidised (i.e. when the fluidised bed height reached above 10 cm), the magnetic stirrer at the bottom of the column is engaged at approx. 80% full speed in order to ensure an even distribution of the incoming liquid and the flow rate is adjusted to 2.2 ml/min. The washing with adsorbent equilibration buffer is continued for 15 min. in which time a stabilised fluidised bed is formed with a fluidised bed height of 16 cm. The stability of the fluidised bed is established by a careful visual inspection of the bed. Following the establishment of a stabilised and equilibrated fluid bed, 300 ml bovine blood is pumped into the column at a steady flow rate of 2.2 ml/min. This is achieved by connecting the tubing through a syringe to a suitable vein in a cow and pumping blood in a continuous process through the column from the bottom inlet and returned to another suitable vein in cow from the top outlet. Small samples of blood are taken from the top outlet each 5 minutes throughout the experiment. The adsorption is run for 1 hour at 5 ml per minute and then terminated. Clotting is avoided by continuously adding a heparin solution in PBS amounting to 25 IU/ml blood through a valve at the bottom inlet of the column.

The results will show if the whole operation can be performed without the occurrence of clotting of the blood, without any extensive cell damage and with no harm to the animal. Furthermore, analysis of the collected outlet-fractions by flow cytometry will demonstrate the extent of which CD8-cells are depleted from the outlet blood stream.

### Example 8

### Bind and Catch Example: The Use of a Stabilised Fluidised Bed Comprising Immunoadsorbent High Density Conglomerate Particles Derivatised with Anti Immunogloblin for the Removal of CDB Positive T-Cells in a Live Host

The purpose and execution of this example is similar to Example 7, except that a CD8-specific antibody is first injected intravenously into a cow as a bolus injection of 20 ml sterile PBS containing 1 mg/ml mouse anti-CD8. This is then followed by extracorporeal adsorption as described in Example 11 to adsorbent particles having anti-mouse immunoglobulin (DAKO Z0109, 3 mg/ml) as attached ligand.

The results will show if the whole operation can be performed without the occurrence of clotting of the blood, without any extensive cell damage and with no harm to the animal. Furthermore, analysis of the collected outlet-fractions by flow cytometry will demonstrate if CD8-cells are depleted from the outlet blood stream with a capacity depending on the volumen of the bed.

### Example 9

### Immobilization of Polymyxin B and Use of Polymyxin B-Coupled Particles for Binding of Bacterial Lipopolysaccharide

The aim of this example is to demonstrate the feasibility of using a Polymyxin B-coupled adsorbent for the binding of LPS.

The adsorbent used for this experiment is a high density divinylsulfone-coupled agarose/stainless steel adsorbent (Upfront Chromatography A/S, Denmark) to which Polymyxin B sulfate (Sigma) is coupled.
Bead composition: epichlorohydrin cross-linked agarose (4% w/v) with a core of stainless steel particles (FIG. 7)
Bead shape: Mainly spherical
Diameter: 20-40 μm
Average individual bead density in the hydrated state: 3.8 g/ml
Ligand: Polymyxin B (Sigma, Mo, code no. P4932) coupled through divinylsulfone at 3 mg/ml. Coupling is performed to achieve coupling of each Polymyxin B molecule through a minimal number of the primary amino groups present in Polymyxin B. Briefly, particles are coupled with Polymyxin B by overnight incubation at room temperature with Polymyxin B in 0.1 M carbonate, 0.5 M NaCl, pH 8.2 at 20 mg/ml, using gentle agitation. Hereafter, particles are washed in the same buffer, and free reactive vinylsulfone groups are blocked with ethanolamine (1 M ethanolamine, pH 9.0, 2 hours at room temperature) and then washed with PBS.
   Procedure:

LPS from E. coli 055:B5 is obtained from Sigma (code no. L2880) and dissolved to 1 μg/ml in milliQ water, yielding a clear solution. The solution is then treated in a batch adsorption process with the Polymyxin B-coupled particles described above. A control experiment is performed using particles derivatised with a non-relevant peptide and blocked with ethanolamine. After incubation for 2 hours under gentle agitation the adsorbent particles are separated from the solution by decanting, and the solution is retrieved for analysis.

Particles are subsequently washed by incubation and decanting with PBS (3 times), collecting each wash separately. The LPS solution before and after treatment as well as all wash solutions are then analysed for LPS with the Limulus amebocyte lysate test and, after freeze-drying and resolubilization directly in sample buffer by sodium dodecyl sulfate polyacrylamide gel electrophoresis (SDS-PAGE) followed by silver staining with an appropiate oxidative treatment ad modum Tsai & Frasch (1982, Anal. Biochem. 119, 115-119).

The results are expected to show that LPS is removed efficiently (to below 0.1 ng/ml) by exposure to the Polymyxin B-coated particles while no difference is expected to be seen with control particles. This is expected to be indicated both by the absence of endotoxin activity in the Limulus amebocyte lysate test and by the absence of LPS-related silver-stained bands in the SDS-PAGE analysis. In a series of similar incubations, varying the ratio between particles and amount of LPS, it is expected that the capacity of the Polymyxin B-derived particles for E. coli 055:B5 LPS can be determined and that it will be high, i.e. exceeding 1 mg LPS pr. 5 ml sedimented bed.

In another series of incubations, different types of LPS will be tested, including LPS from Salmonella Typhimurium and from rough E. coli types (short-chain LPS), and the results are expected to show that these types of LPS are also bound efficiently and with high capacity to the Polymyxin B-coated particles. During these experiments the elution of bound LPS and the reuse of the particles will also be attempted and it is expected that reusage of the particles will be possible, using buffer condittions similar to those described by Issekutz (1983, “Removal of gram-negative endotoxin from solutions by affinity chromatography”, J. Immunol. Meth. 61, 275-281) by washing with detergent, followed by extensive washings with saline.

### Example 10

### Use of Polymyxin B-Coupled Particles in a Stabilised Fluidised Bed for the Adsorption of LPS in Whole Blood

The aim of the following experiment is to establish the feasibility of binding of LPS in whole human blood by Polymyxin B-coated particles in a stabilised fluidised bed procedure.

Materials and Methods:

The experimental set-up and equipment used was the same as in Example 1.

Adsorbent Particles (with Polymyxin B as Ligand):

The adsorbent used for this experiment is a high density divinylsulfone-coupled agarose/stainless steel adsorbent (Upfront Chromatography A/S, Denmark) to which Polymyxin B sulfate (Sigma) is coupled.
Bead composition: epichlorohydrin cross-linked agarose (4% w/v) with a core of stainless steel particles (FIG. 7)
Bead shape: Mainly spherical
Diameter: 20-40 μm
Average individual bead density in the hydrated state: 3.8 g/ml
Ligand: Polymyxin B (Sigma, Mo, code no. P4932) coupled through divinylsulfone at 3 mg/ml. Coupling is performed to achieve coupling of each Polymyxin B molecule through a minimal number of the primary amino groups present in Polymyxin B, see Example 9.
   Adsorbent Equilibration Buffer:

6% w/v dextran MW 110.000 (Pharmacosmos, Denmark) in 0.9% w/v sodium chloride was used to pre-equilibrate the adsorbent before percolation of the blood through the column.

Blood:

A freshly drawn human blood sample from a healthy donor, collected in standard EDTA glass tubes (Becton Dickinson, code no. 15067), was used for the experiment. The blood was stored at room temperature and used within 1 hour after collection. Just prior to the adsorption procedure, 100 μg E. coli 055:B5 LPS (Sigma, MO, L2880) is added to 100 ml of the blood to give a final concentration of 1 μg LPS per ml human blood.

Procedure:

The fluid bed column is assembled according to the supplier's instructions and added an aqueous suspension of the adsorbent particles to reach a sedimented bed height of 5.8 cm.

In order to ensure an optimal salt concentration/osmolality for minimal hemolysis of the blood cells when entering the column, a wash with adsorbent equilibration buffer is initially performed at a flow rate of 2.2 ml/min. When the adsorbent particles are fluidised by the upward flow of equilibration buffer the magnetic stirrer is engaged at 80% full speed and The column is positioned carefully to a completely vertical state. When reaching a fully stabilised fluidised state, the height of the adsorbent bed is expected to be increased to 10.5 cm.

Following the initial wash with equilibration buffer, the blood sample is applied to the column with a flow rate of 2.2 ml/min. A well-defined weakly parabolic front of blood is then observed moving gradually up through the stabilised fluidised bed. No back mixing or channelling is expected to be observed throughout the experiment.

Following the application of the blood sample, the entire run-through is collected and the column is washed with 200 ml adsorbent equilibration buffer in order to wash out the blood and any unbound macromolecules therein. This wash fraction is also collected. In a parallel set-up, a control column containing particles derivatised with a non-relevant peptide is used for treating LPS-spiked blood in the same way as described above.

All run-through and wash fractions are analysed by the Limulus amebocyte lysate assay.

Result:

After passage of the LPS-spiked blood through the stabilised fluidised bed of Polymyxin B-coated particles it is expected that all LPS are removed from the blood with a minimal degree of hemolysis occurring in the blood (expected to be below 0.2% as determined by spectrophotometry at 540 nm) while no LPS is expected in the wash fractions. No absorption of LPS is expected to be seen with the control column as all LPS is still expected to be found in the run-through fraction; in this case, while the LPS-activity as measured by the Limulus amebocyte lysate assay may be found to be decreased due to a certain amount of LPS-neutralising activity in blood, the difference between the control adsorbent and the Polymyxin B-adsorbent is expected to be very clear. SDS-PAGE is not directly applicable for analysis of trace concentrations of LPS in blood due to the presence of large amounts of cells and serum proteins.

It is furthermore expected that the Polymyxin-B adsorbent can be reused after elution of bound LPS by 1% sodium deoxycholate in 0.1 M Tris, pH 8, followed by extensive washings with saline (as taught by Issekutz, 1983, “Removal of gram-negative endotoxin from solutions by affinity chromatography”, 3. Immunol. Meth. 61, 275-281).

### Example 11

### Prevention of Endotoxicosis in a Bovine Model, by Extracorporeal Adsorption of Blood from LPS-Challenged Cows on a Stabilised Fluidised Bed of Polymyxin B-Containing Particles

The aim of this experiment is to demonstrate the ability of the extracorporeal adsorption process of the present invention to remove LPS from the circulating blood of a whole animal to a degree leading to a significant reduction in clinical signs.

It should be remembered, however that this example demonstrates the “detoxifying” potential of the invention in the context of an animal being subjected to a bolus injection of pure LPS; this may be quite different from the situation in a mammal experiencing sepsis, in which instead LPS must be expected to enter the blood circulation in a continuous mode and starting at relatively low levels, depending on the development of the underlying infection and it's association with the blood stream.

Clinically healthy, non-lactating Danish Holstein cows weighing from 500 to 800 kg is included in the study. These animals is challenged by intravenous injection of 1000 ng LPS/kg body weight (E. coli 055:B5 LPS, Sigma, L2880) through a catheter in vena auriculis intermedia or v. auriculis medialis passed through v. auricularis caudalis to v. jugularis externa. Such a challenge is normally followed by pronounced host responses, including increased rectal temperature and heart rate within the first 3-24 hours after challenge (Tølbøll, T. H., 2002, “Bovine endotoxicosis”, ph.d. thesis, Royal Veterinary Agricultural University, Denmark) but does not lead to endotoxic shock.

By applying a venous-venous extracorporeal adsorption circuit, comprising a stabilised fluidised bed of Polymyxin B-coated particles to a cow being challenged with LPS as described above, this experiment is intended to show the effect of removing LPS from the circulation at different times after its intravenous injection. To do this, clinical parameters, including rectal temperature, heart rate, respirtory frequency, and acute phase protein responses will be measured up to one week after the challenge and compared between cows treated by the described extracorporeal method and cows not treated. Also, the effect of this extracorporeal treatment on the clinical outcome of increasing doses of LPS will be studied.

Results are expected to show that LPS-challenged cows treated with extracorporeal adsorption of the animal's blood in a continuous process through a stabilised fluidised bed of Polymyxin B-coated particles present with significantly less, significantly less severe and significantly more short-lived clinical signs than comparable, non-treated cows. It is also an expected result that the treatment is efficient even when applied some time after the LPS-challenge, for example up to 12 hours after the LPS-challenge.

### Example 12

### Immobilization of Toll-Like Receptor 4 (TLR4) and Use of TLR4-Coupled Particles for Binding of Bacterial Lipopolysaccharide

The aim of this example is to demonstrate the feasibility of using a TLR4-coupled adsorbent for the binding of LPS.

The adsorbent used for this experiment is a high density divinylsulfone-coupled agarose/stainless steel adsorbent (Upfront Chromatography A/S, Denmark) to which TLR4 is coupled.
Bead composition: epichlorohydrin cross-linked agarose (4% w/v) with a core of stainless steel particles (FIG. 7)
Bead shape: Mainly spherical
Diameter: 20-40 μm
Average individual bead density in the hydrated state: 3.8 g/ml
Ligand: TLR4 coupled through divinylsulfone at 3 mg/ml. Coupling is performed to achieve coupling of each TLR4 molecule through a minimal number of the primary amino groups present in TLR4. Briefly, particles are coupled with TLR4 by overnight incubation at room temperature with TLR4 in 0.1 M carbonate, 0.5 M NaCl, pH 8.2 at 20 mg/ml, using gentle agitation. Hereafter, particles are washed in the same buffer, and free reactive vinylsulfone groups are blocked with ethanolamine (1 M ethanolamine, pH 9.0, 2 hours at room temperature) and then washed with PBS.
   Procedure:

LPS from E. coli 055:B5 is obtained from Sigma (code no. L2880) and dissolved to 1 μg/ml in milliQ water, yielding a clear solution. The solution is then treated in a batch adsorption process with the TLR4-coupled particles described above. A control experiment is performed using particles derivatised with a non-relevant peptide and blocked with ethanolamine. After incubation for 2 hours under gentle agitation the adsorbent particles are separated from the solution by decanting, and the solution is retrieved for analysis. Particles are subsequently washed by incubation and decanting with PBS (3 times), collecting each wash separately. The LPS solution before and after treatment as well as all wash solutions are then analysed for LPS with the Limulus amebocyte lysate test and, after freeze-drying and resolubilization directly in sample buffer by sodium dodecyl sulfate polyacrylamide gel electrophoresis (SDS-PAGE) followed by silver staining with an appropiate oxidative treatment ad modum Tsai & Frasch (1982, Anal. Biochem. 119, 115-119).

The results are expected to show that LPS is removed efficiently (to below 0.1 ng/ml) by exposure to the TLR4-coated particles while no difference is expected to be seen with control particles. This is expected to be indicated both by the absence of endotoxin activity in the Limulus amebocyte lysate test and by the absence of LPS-related silver-stained bands in the SDS-PAGE analysis. In a series of similar incubations, varying the ratio between particles and amount of LPS, it is expected that the capacity of the TLR4-derived particles for E. coli 055:B5 LPS can be determined and that it will be high, i.e. exceeding 1 mg LPS pr. 5 ml sedimented bed.

In another series of incubations, different types of LPS will be tested, including LPS from Salmonella Typhimurium and from rough E. coli types (short-chain LPS), and the results are expected to show that these types of LPS are also bound efficiently and with high capacity to the TLR4-coated particles. During these experiments the elution of bound LPS and the reuse of the particles will also be attempted and it is expected that reusage of the particles will be possible, using buffer condittions similar to those described by Issekutz (1983, “Removal of gram-negative endotoxin from solutions by affinity chromatography”, J. Immunol. Meth. 61, 275-281) by washing with detergent, followed by extensive washings with saline.

It is expected that the TLR4-coupled particles can be used as described in Examples 10 and 11 above for the adsorption of LPS in whole blood and for the prevention of endotoxicosis in a bovine model.

### Example 13

### The Use Extracorporeal Adsorption for the Treatment of Endotoxin-Challenged Cows by Placing a Stabilised Fluidised Bed of LPS-Binding Particles in Line with a Switch being Activated when a Blood Biomarker Reaches a Certain, Critical Value

The aim of this experiment is to show the possibility of subjecting a sepsis-prone animal to a surveillance system consisting of the following components:
1) a device for continuous monitoring of blood concentration of selected analytes placed in line with the blood circulation of the animal. Said device will send an signal activating (opening) a switch when the blood concentration of the selected analyte reaches a pre-set, non-normal level
2) a switch capable of being activated by the monitoring device
3) a stabilised fluidised bed as described in this invention placed in line with the blood circulation when the switch is activated and cut off from the blood circulation when the switch is not activated.

It should be remembered, however that this example demonstrates the “detoxifying” potential of the invention in the context of an animal being subjected to a bolus injection of pure LPS; this may be quite different from the situation in a mammal experiencing sepsis, in which instead LPS must be expected to enter the blood circulation in a continuous mode and starting at relatively low levels, depending on the development of the underlying infection and it's association with the blood stream.

Clinically healthy, non-lactating Danish Holstein cows weighing from 500 to 800 kg will be included in the study. These animals will be challenged by intravenous injection of 1000 ng LPS/kg body weight (E. coli 055:B5 LPS, Sigma, L2880) through a catheter in vena auriculis intermedia or v. auriculis medialis passed through v. auricularis caudalis to v. jugularis externa. Such a challenge is normally followed by pronounced host responses, including increased rectal temperature and heart rate within the first 3-24 hours after challenge (Tølbøll, T. H., 2002, “Bovine endotoxicosis”, ph.d. thesis, Royal Veterinary Agricultural University, Denmark) but does not lead to endotoxic shock.

Shortly after the injection of LPS the cow is connected to a venous-venous extracorporeal adsorption circuit, comprising a stabilised fluidised bed of Polymyxin B-coated particles connected via a switch, this switch being activated by a continuous monitoring device, detecting changes in the serum concentration of haptoglobin in the blood. The cow is being challenged with LPS as described above.

The experiment is intended to show the effect of removing LPS from the circulation by the stand-by extracorporeal adsorption circuit. Clinical parameters, including rectal temperature, heart rate, respirtory frequency, and acute phase protein responses will be measured up to one week after the challenge and compared between cows treated by the described extracorporeal method and cows not treated. Also, the effect of this extracorporeal treatment on the clinical outcome of increasing doses of LPS will be studied.

Results are expected to show that LPS-challenged cows treated by stand-by extracorporeal adsorption of the animal's blood in a continuous process through a stabilised fluidised bed of Polymyxin B-coated particles present with significantly less, significantly less severe and significantly more short-lived clinical signs than comparable, non-treated cows. It is also an expected result that the treatment is more efficient than a treatment applied at a fixed, later time after the LPS-challenge, for example 12 hours after the LPS-challenge.

1. An extracorporeal adsorption method for removing harmful substances responsible of inducing sepsis caused by Gram-negative in a mammal, said extracorporeal adsorption method being effected by an adsorption column assembly, said adsorption column assembly comprising a column and an adsorption medium in the form of particles, the sedimented volume of said particles being at the most 80% of the volume of the column, said particles being characterised by carrying an affinity specific molecule with a specific affinity for the LPS portion of said Gram-negative bacteria, said method comprising treating blood obtained from said mammal by passing the blood through the adsorption column assembly at such a flow rate that a fluidised bed of the particles is formed.

2. An extracorporeal adsorption method for removing harmful substances responsible of inducing sepsis caused by Gram-negative or Gram-positive bacteria in a mammal, said extracorporeal adsorption method being effected by an adsorption column assembly, said adsorption column assembly comprising a column and an adsorption medium in the form of particles, the sedimented volume of said particles being at the most 80% of the volume of the column, said particles being characterised by carrying an affinity specific molecule with a specific affinity for:
i) the LPS portion of said Gram-negative bacteria, and/or
ii) Gram-positive bacteria or harmful substances derived from said Gram-positive bacteria, said method comprising treating blood obtained from said mammal by passing the blood through the adsorption column assembly at such a flow rate that a fluidised bed of the particles is formed.

3. A method according to claim 1 wherein the treated blood is capable of being reinfused into the same mammal.

4. A method according to claim 1, wherein the adsorption column assembly is adapted for fluidised bed adsorption, in particular stabilised fluidised bed adsorption.

5. A method according to claim 1, wherein the particles have a density of at least 1.3 g/ml and a mean diameter in the range of 5-1000 μm, such as a density of at least 1.5 g/ml and a mean diameter in the range of 5-300 μm, preferably a density of at least 1.8 g/ml and a mean diameter in the range of 5-150 μm, and most preferred a density of more than 2.5 g/ml and a mean diameter in the range of 5-75 μm.

6. A method according to claim 1, wherein the mammal is a human being.

7. A method according to claim 1, wherein the affinity specific molecule is selected from the group consisting of immunoglobulins, peptides, oligonucleotides, receptor proteins, antibiotics, and lectins.

8. A method according to claim 1, wherein two or more different affinity specific molecules are present on particles within the adsorption medium.

9. A method according to claim 6, wherein the affinity specific molecules are selected from immunoglobulins.

10. A method according to claim 1, wherein the affinity specific molecule is Polymyxin B.

11. A method according to claim 1, wherein the affinity specific molecule is selected from the group consisting of a Toll-like receptor, most preferably TLR4 or binding fragments thereof or multimeric arrangements thereof, CD14, MD2, TLR2 and LBP, and any combination thereof.

12. A method according to claim 1, wherein the sedimented volume of the particles is at the most 70% of the volume of the column, such as at the most 60% of the volume of the column, e.g. at the most 50% of the volume of the column.

13. Use of an adsorption medium for the preparation of a therapeutic adsorption column assembly for the continues therapeutic treatment of sepsis caused by Gram-negative bacteria in a mammal by extracorporeal adsorption, said adsorption column assembly comprising (i) a vessel for continues obtaining blood from said mammal, (ii) a column comprising the adsorption medium, the sedimented volume of said adsorption medium being at the most 80% of the volume of the column, said adsorption medium being characterised by carrying an affinity specific molecule with a specific affinity for the LPS portion of said Gram-negative bacteria, said column is treating the obtained blood by passing the blood through the adsorption column assembly at such a flow rate that a fluidised bed of the adsorption medium is formed, and (iii) another vessel which continuously delivers blood back to the patient.

14. Use of an adsorption medium for the preparation of a therapeutic adsorption column assembly for the therapeutic treatment of sepsis caused by Gram-negative or Gram-postive bacteria in a mammal by extracorporeal adsorption, said adsorption column assembly comprising (a) a vessel for continuos obtaining blood from said mammal, (b) a column and the adsorption medium, the sedimented volume of said adsorption medium being at the most 80% of the volume of the column, said adsorption medium being characterised by carrying an affinity specific molecule with a specific affinity for:
i) the LPS portion of said Gram-negative bacteria, and/or
ii) Gram-positive bacteria or harmful substances derived from said Gram-positive bacteria, said column is treating the obtained blood by passing the blood through the adsorption column assembly at such a flow rate that a fluidised bed of the adsorption medium is formed, and
(c) another vessel which continuously delivers blood back to the patient.

15. The use according claim 13, wherein the flow rate of the blood through the column assembly is such that expansion ratio of the fluidised bed is at least 1.3, such as at least 1.5.

16. The use according to claim 12, wherein the steps (a), (b) and (c) are preceded by a initial step by which a substance is first injected into the blood stream of the mammal.

17. The use according to claim 13, wherein the mammal is a human being.

18. The use according to claim 13, wherein the particles have a density of at least 1.3 g/ml and a mean diameter in the range of 5-1000 μm, such as a density of at least 1.5 g/ml and a mean diameter in the range of 5-300 μm, preferably a density of at least 1.8 g/ml and a mean diameter in the range of 5-150 μm, and most preferred a density of more than 2.5 g/ml and a mean diameter in the range of 5-75 μm.

19. A use according to claim 11, wherein the stabilised fluidised bed is placed in line with a switch capable of being activated when a blood substance reaches a pre-set value, said blood substance is monitored by a device, said device is placed in line with the blood circulation, said device sending the activating signal to the switch when said value is reached.

20. The use according to claim 13, wherein the affinity specific molecule is selected from the group consisting of immunoglobulins, peptides, oligonucleotides, receptor proteins, antibiotics, and lectins.

21. The use according to claim 13, wherein two or more different affinity specific molecules are present on particles within the adsorption medium.

22. The use according to claim 20, wherein the affinity specific molecules are selected from immunoglobulins.

23. The use according to claim 20, wherein the affinity specific molecule is Polymyxin B.

24. A use according to claim 13, wherein the affinity specific molecule is selected from the group consisting of a Toll-like receptor, most preferably TLR4 or binding fragments thereof or multimeric arrangements thereof, CD14, MD2, TLR2 and LBP, and any combination thereof.

25. The use according to claim 13, wherein the sedimented volume of the particles is at the most 70% of the volume of the column, such as at the most 60% of the volume of the column, e.g. at the most 50% of the volume of the column.

26. The use according to claim 13, wherein the flow rate is such that stabilised fluidised bed of the particles is formed.
